(19) United States
(12) Patent Application Publication
(10) Pub. No.: US 2013/0184168 A1
(43) Pub. Date: Jul. 18, 2013

(54) IMMUNOREACTIVE FRANCISELLA TULARENSIS ANTIGENS

(71) Applicants: Mary Katherine HART, Frederick, MD (US); Robert Victor HOUSE, Frederick, MD (US); Shannon MARTIN, Frederick, MD (US)

(72) Inventors: Mary Katherine HART, Frederick, MD (US); Robert Victor HOUSE, Frederick, MD (US); Shannon MARTIN, Frederick, MD (US)

(21) Appl. No.: 13/350,233

(22) Filed: Jan. 13, 2012

(51) Int. Cl. C40B 30/04; G01N 33/566; C07H 19/207

(52) U.S. Cl. 506/9; 536/26.23; 435/7.92

(57) ABSTRACT

The present invention relates to immunoreactive Francisella tularensis antigens and their uses as correlates of protection against tularemia. In one aspect, the invention provides a set of biomarkers for tularemia. In another aspect the invention provides a method of evaluating immunity against tularaemia in a subject, using the biomarkers.

### FIELD OF THE INVENTION

The present invention relates to immunoreactive Francisella tularensis antigens and uses thereof. More specifically, the invention relates to immunoreactive Francisella tularensis antigens and their uses as correlates of protection.

### BACKGROUND OF THE INVENTION

Tularemia is a disease caused by the Gram-negative facultative intracellular bacterium, Francisella tularensis. F. tularensis is pathogenic for many mammalian species including humans, causing a spectrum of diseases collectively called tularemia. Tularemia has been reported as a clinical infection in primates in many temperate climates across the world. Several subspecies exist, with the most clinically relevant subspecies being holarctica and tularensis, commonly denoted Type B and A strains, respectively (Sjostedt, 2001). The subspecies tularensis (Type A) is endemic only to North America. Mortality rates of up to 60% have been reported for untreated human cases of disseminated infection caused by Type A strains of the pathogen (Dienst, 1963). The subspecies holarctica (Type B), endemic to both Europe and North America, is associated with a less severe clinical manifestation and lower mortality rates. Type B strains are responsible for almost all European cases of tularemia (Sjostedt, 2007).

In the 1950's a live vaccine strain (LVS) was empirically derived from a Soviet strain, S15, and was found to protect humans to some degree against subsequent exposure to Type A strains of the pathogen (Hornick & Eigelsbach, 1966). For example, when LVS replaced killed bacteria as the vaccine at USAMRIID, the incidence of respiratory infections among at risk personnel was significantly reduced (Burke, 1977; Eigelsbach et al, 1967). Human volunteer LVS vaccination studies were conducted under the Operation Whitecoat (OW) program in the 1950s. These data showed that LVS administered by scarification was 25-100% effective against aerosol challenge with SCHU S4 (Hornick and Eigelsbach, 1966). In addition, all vaccinees were shown to seroconvert to an undefined set of Francisella antigens, but no immunologic correlation was established with the protective status of the host.

Due to renewed concerns regarding its potential use in bioterrorism, there has been an increased interest during the past decade in licensing a tularemia vaccine for general use. LVS remains the only tularemia vaccine to have shown efficacy in humans. LVS has been successfully used in Europe and the USA to protect tularemia researchers against Type A strains (Oyston et al, 2005; Conlan, 2004; Titball & Oyston, 2003). The LVS NDBR101 Lot 11 has also been administered to Swedish laboratory staff, and during four decades of active tularemia research, there have been very few reported cases of laboratory-acquired infections in vaccinated individuals. However, the absence of a correlate of protection is one of several significant barriers to the licensure of LVS.

During OW and subsequent studies, no correlation between the antibody titre to protein antigens in humans and level of protection against challenge with virulent F. tularensis was observed (Hornick & Eigelsbach, 1966; Saslaw & Carhart, 1961; Saslaw & Eigelsbach, 1961a; Saslaw & Eigelsbach, 1961b). In the past decade, a handful of studies have surveyed the repertoire of murine antibodies generated in response to LVS vaccination (Havlasova et al, 2005; Sundaresh et al, 2007; Eyles et al, 2007) and human tularemia infection (Havlasova et al, 2002; Janovska et al, 2007a; Janovska et al, 2007b). Some of these studies measured only antibody titres in the subjects, while others used immunoproteomics on sera from LVS infected mice; however, the applicability of findings in animal models, especially mice, in humans is questionable.

Ethical considerations prevent tularaemia vaccine efficacy studies in humans; thus, evaluation of vaccines must be conducted in animal models of tularaemia using the FDA Animal Rule. The Animal Rule allows demonstration of the efficacy of vaccines that cannot be tested in human clinical trials via efficacy studies in animals. Application of the Animal Rule to tularaemia vaccine candidates would be facilitated by immunological correlate of protection or vaccine marker to bridge efficacy in animals to immunogenicity in humans.

Thus, there remains a need in the art for a correlate of protection to establish a positive relationship between efficacy of tularaemia vaccine in animals and humans.

### SUMMARY OF THE INVENTION

The present invention relates to immunoreactive Francisella tularensis antigens and uses thereof. More specifically, the invention relates to immunoreactive Francisella tularensis antigens and their uses as correlates of protection.

Thus, the present invention provides a set of biomarkers for tularaemia, selected from the group consisting of: dihydrolipoamide succinyltransferase component of 2-oxoglutarate dehydrogenase complex, 50S ribosomal protein L7/L12, 30S ribosomal protein S1, DNA-directed RNA polymerase alpha subunit, Acetyl-CoA carboxylase, Outer membrane associated protein FopA, Peroxidase/catalase, Chaperone protein DnaK, Pyruvate dehydrogenase E2 component, Chaperone protein groEL, Hypothetical membrane protein, and any combination thereof. The biomarkers of the present invention may be reactive with tularaemia-exposed sera at a frequency of about 30-100%.

The present invention also provides a method of evaluating immunity against tularaemia in a subject, comprising:
a. contacting serum from the subject with the set of biomarkers of the present invention;
b. evaluating immunoreactivity of the serum to the biomarkers;
c. determining the protection against tularaemia based on immunoreactivity to the biomarkers.

The present invention further provides a method of evaluating vaccine efficacy, comprising:
a. contacting serum from a vaccinated model of tularaemia with the biomarkers of the present invention;
b. evaluating immunoreactivity of the serum to the biomarkers;
c. correlating the immunoreactivity to immunoprotective status of the model; and
d. predicting vaccine efficacy in human based on the correlation of step c.

Recent studies in the murine model of tularemia show that adaptive host defense against F. tularensis is likely mediated by both cell-mediated immunity (CMI) and humoral immunity (Tarnvik, 1989; Elkins et al, 2003; Kirimanjeswara et al, 2008). Although CMI is thought to be the most essential mechanism in host defense against Type A Francisella, specific antibody responses are mounted during natural Francisella infections or following vaccination (Dennis et al, 2001; Saslaw & Carhart, 1961; Carlsson et al, 1979; Viljanen et al, 1983). Patients that recover from types A and B Francisella infections are rarely reported to show signs of disease following a second exposure, and therefore could be considered a group that is protected from further challenge. Therefore, this allows the comparison of the repertoire of antibodies between infected, but presumably protected individuals, and vaccinated volunteers whose protective status is unknown.

The present invention makes use of immunoproteomics to identify the antigenic proteins from human tularemia patients and LVS vaccinees, including sera from subjects in FDA clinical trial of LVS. A gel-based immunoproteomics approach was used to identify immunoreactive proteins generated in response to LVS vaccination of mice, rabbits, non-human primates (NHP), and humans. Proteins observed to be immunoreactive with the majority of sera within a study group or across species were identified by tandem mass spectrometry. The results showed that tularaemia infection or LVS vaccination stimulates the generation of antibodies towards a small subset of the Francisella proteome. Specifically, eleven proteins were downselected as commonly reactive antigens, reactive with both patient and vaccinee sera, with a minimum frequency of 30% (i.e., proteins were observed to be reactive with minimum 30% of sera screened). These include dihydrolipoamide succinyltransferase component of 2-oxoglutarate dehydrogenase complex (FT_{—}0077), 50S ribosomal protein L7/L12 (FTT_{—}0143), 30S ribosomal protein S1 (FTT_{—}0183), DNA-directed RNA polymerase alpha subunit (FTT_{—}0350), Acetyl-CoA carboxylase (FTT0472), Outer membrane associated protein FopA (FTT_{—}0583), Peroxidase/catalase FTT_{—}0721c), Chaperone protein DnaK (FTT_{—}1269c), Pyruvate dehydrogenase E2 component (FTT_{—}1484c), Chaperone protein groEL (FTT1696), Hypothetical membrane protein (FTT_{—}1778c). Similarities were observed in the repertoire of immunoreactive proteins generated by LVS vaccination across species. No single immunoreactive proteins correlating with protection of the host; however, a combination of the immunoreactive proteins or a qualitative immune response to several immunogenic proteins may provide a correlate of protection.

Additional aspects and advantages of the present invention will be apparent in view of the following description. The detailed description and examples, while indicating preferred embodiments of the invention, are given by way of illustration only, as various changes and modifications within the scope of the invention will become apparent to those skilled in the art in light of the teachings of this invention.

### BRIEF DESCRIPTION OF THE DRAWINGS

These and other features of the invention will now be described by way of example, with reference to the appended drawings, wherein:

FIG. 1 shows Western blots probed with sera from Type B patients. 100 μg of SCHU S4 Δwbtl (O-antigen negative) was used as the antigen, with first dimension separation in the pH range 4-7. Blots were probed with 1:500 dilution of human sera.

FIG. 2 shows Western blots probed with sera from Type A patients. Patient identification numbers are indicated, above each blot. 100 μg of SCHU S4 Δwbtl (O-antigen negative) was used as the antigen, with first dimension separation in the pH range 4-7. Blots were probed with 1:500 dilution of human sera.

FIG. 3 shows Western blots probed with sera from LVS vaccinees (Umea). Patient identification numbers are indicated above each blot image. D0, indicates sera drawn on the day of vaccination, d42 denotes sera drawn 42 days post vaccination. 100 μg of SCHU S4 Δwbtl (O-antigen negative) was used as the antigen, with first dimension separation in the pH range 4-7. Blots were probed with 1:500 dilution of human sera.

FIG. 4 shows Western blots probed with sera from LVS vaccinaees (Baylor). 100 μg of SCHU S4 Δwbtl (O-antigen negative) was used as the antigen, with first dimension separation in the pH range 4-7. Blots were probed with 1:500 dilution of human sera.

FIG. 5 shows representative Two-dimensional Western blots probed with sera from tularemia patients and LVS vaccinees. 100 μg of SCHU S4 Δwbtl (O-antigen negative) was used as the antigen, with first dimension separation in the pH range 4-7. Blots were probed with 1:500 dilution of human sera as follows (a) Control 1, (b) Type B tularemia patient serum number 1671, (c)) Type A tularemia patient serum number MV758, (d) Type A tularemia patient serum number MV756, (e) NDBR lot 11 LVS vaccinee control serum number 110d0, (f) NDBR lot 11 LVS vaccinee day 42 post vaccination serum number 201d42, (g) DVC lot 17 LVS vaccine pre-vaccination serum number 200139603 (paired id 02832), (h)) DVC lot 17 LVS vaccine day 42 post-vaccination serum number 2001362794 (paired id 02832). The complete set of Western blot images are shown in supplementary data FIGS. 1-4.

FIG. 6 shows a two-dimensional gel resolving the SCHU S4 proteome. Protein stained 2D-gel, separating SCHU S4 protein lysates in the pH range 4-7, used for alignment of 2D Western blots and identification of immunoreactive proteins. Identified proteins are annotated with their locus tags, and listed in full in Table 2.

FIG. 7 is a summary of immunoreactive proteins. FIG. 7A shows the immunoreactive proteins and their relative intensity values observed for each serum screened. The bar chart below this matrix shows the total intensity of the immunoreactive proteins for each serum sample screened. FIG. 7B shows the predicted subcellular locations for the identified immunoreactive proteins. These were determined using the PSORT1b algorithm, as described in the methods. FIG. 7C shows the Clusters of Orthologous Group classification of the identified immunoreactive proteins.

FIG. 8 is a bar chart representation of the frequency with which each immunoreactive protein was observed in the sera screened. Square fill indicates immunoreactive proteins that were only observed to react with sera from tularemia patients. Vertical line fill indicates proteins that were observed to be immunoreactive only with sera from LVS vaccinees. Black fill indicates immunoreactive proteins that were observed to be reactive with sera from both tularemia patients and vaccinees.

FIG. 9 shows representative 2D Western blots probed with sera from LVS vaccinated non-human primates (Rhesus macaque LVS efficacy vaccination study). Shown are representative 2D-Western blots probed with sera from animal 0607036, seven days pre-LVS vaccination (FIG. 9A); animal 0607036, 28 days post-LVS vaccination (scarification) (FIG. 9B); animal 0602084, 28 days post LVS vaccination (subcutaneous) (FIG. 9C); animal RQ7796 56 days post LVS vaccination (scarification) (FIG. 9D); and animal RQ7459, 56 days post LVS vaccination (subcutaneous) (FIG. 9E). The complete set of blots, including pre and post vaccination for each animal, are shown in FIG. 10.

FIG. 10 shows 2D-Western blots of sera from LVS-vaccinated non human primate. Group 3 (FIG. 10A) animals were vaccinated via scarification and challenged 35 days post-vaccination. Group 4 (FIG. 10B) animals were vaccinated subcutaneously and challenged 35 days post-vaccination. Group 5 (FIG. 10C) animals were vaccinated via scarification and challenged 63 days post-vaccination. Group 6 (FIG. 10D) animals were vaccinated subcutaneously and challenged 63 days post-vaccination. Sera are paired, with the left hand blot of each pair probed with pre-vaccinated sera and the right hand blot of each pair probed with sera drawn post-vaccination.

FIG. 11 is a matrix of immunoreactive proteins for non-human primate LVS efficacy study. The blot images were analyzed using PDQuest software and the comparative intensity values for each immunoreactive area measured. Shading indicates the degree of observed immunoreactivity, from no observed immunoreactivity (□), to intense immunoreactivity (▪). The animal numbers shaded in grey indicate LVS vaccinated survivors of SCHU S4 challenge.

FIG. 12 is a bar chart showing total intensity of observed immunoreactivity for LVS vaccinated non-human primates. Animals are grouped by route of vaccination, and by day sera was collected post vaccination. Asterisk (*) indicates animals that survived challenge.

FIG. 13 shows 2D Western blots probed with sera from LVS-vaccinated rabbits. New Zealand White rabbits Animal number 7, sera drawn seven days pre-vaccination (FIG. 13A); Animal number 7, sera drawn 42 days post LVS vaccination (scarification) (FIG. 13B); Animal number 36, sera drawn 42 days post LVS vaccination (subcutaneous) (FIG. 13C); Animal number 26, sera drawn 63 days post LVS vaccination (subcutaneous) (FIG. 13D); and Animal number 19, sera drawn 63 days post LVS vaccination (scarification) (FIG. 13E).

FIG. 14 shows 2D-Western blots of sera from LVS-vaccinated rabbits. Group 1 animals were subcutaneously vaccinated with LVS and challenged on day 42 with SCHU S4 (FIG. 14A). Group 2 animals were vaccinated with LVS by scarification and challenged on day 42 with SCHU S4 (FIG. 14B). Group 3 animals were sham-vaccinated by scarification (right) or subcutaneously (left, in box) with saline and sera drawn 42 days post vaccination (FIG. 14C). For FIGS. 14A-C, boxed sera are paired, with the left hand blot probed with sera drawn pre-vaccination and the blot on the right probed with sera drawn 42 days post-vaccination; remaining sera (where applicable) are unpaired and were probed with sera drawn 42 days post-vaccination. Group 4 animals were subcutaneously vaccinated with LVS and challenged on day 63 with SCHU S4 (FIG. 14D). Group 5 animals were vaccinated with LVS by scarification and challenged on day 63 with SCHU S4 (FIG. 14E). For FIGS. 14D-E, sera are paired, with the left hand blot in each pair having been probed with sera drawn pre-vaccination, and the blot on the right probed with sera drawn 63 days post-vaccination. Group 6 animals were sham-vaccinated by scarification (top) or subcutaneously (bottom) with saline and sera drawn 63 days post vaccination (FIG. 14F). Group 7 of the LVS-vaccinated rabbit study is shown in FIG. 14G. These blots (FIGS. 14F-G) were probed with sera from control animals

FIG. 15 is a matrix of immunoreactive proteins for LVS-vaccinated rabbits. Shading indicates the degree of observed immunoreactivity, from no observed immunoreactivity (□), to intense immunoreactivity (▪). * denotes animals that survived aerosol challenge with SCHU S4.

FIG. 16 shows characteristics of immunoreactive proteins identified from Western blotting with sera from LVS-vaccinated rabbits. FIG. 16A is a bar chart representation of the frequency with which each immunoreactive protein was observed in the LVS-vaccinated rabbit sera screened. Black fill indicates immunoreactive proteins observed to react with sera from rabbits LVS-vaccinated by scarification. Grey fill indicates immunoreactive proteins reacting with sera from rabbits vaccinated SC with LVS. FIG. 16B is a bar chart showing total intensity of observed immunoreactivity for LVS-vaccinated rabbits. Animals are grouped by route of vaccination, and by day sera was collected post-vaccination. Asterisk (*) indicates animals that survived SCHU S4 challenge.

FIG. 17 shows 2D Western blots probed with sera from mice vaccinated subcutaneously or intranasally with LVS. BALB/c mice were immunised with LVS subcutaneously with lot 17 LVS, and sera drawn at 4 weeks post-vaccination (Mouse 1.1; FIG. 17A), or intranasally 4 weeks post-vaccination (Mouse 4.1; FIG. 17B). Antigen was 100 μg SCHU S4 Δwbtl, separated in the pH range 4-7. Primary sera were used in a 1:500 dilution.

FIG. 18 shows 2D Western blots probed with sera from mice vaccinated subcutaneously with LVS or LVS ΔIgIC mutant. Western blots probed with sera from sham-vaccinated BALB/c mice (FIG. 18A), LVS-vaccinated BALB/c mice (FIG. 18B), and BALB/c mice vaccinated with LVS ΔIgIC mutant (FIG. 18C). Sera were drawn at 4 weeks post-vaccination. Representative blots are shown. Antigen was 100 μg SCHU S4 Δwbtl, separated in the pH range 4-7. Primary sera were used in a 1:500 dilution.

FIG. 19 shows the characteristics of immunoreactive proteins in successful intradermal LVS vaccination with unsuccessful LVS ΔigIC vaccination of BALB/c mice. FIG. 19A is a matrix of immunoreactive proteins, showing results of 2D Western blots probed with sera from mice vaccinated subcutaneously with LVS or LVS ΔIgIC mutant. FIG. 19B is a bar chart showing total observed intensity in immunoreactive spots for each serum screened. FIG. 19C is a bar chart showing observed immunoreactivity for areas corresponding to the proteins SucB/RspA. FIG. 19D is a bar chart showing observed immunoreactivity for areas corresponding to the protein peroxidase/catalase. FIG. 19E is a bar chart showing observed immunoreactivity for areas corresponding to the protein GroEL. FIG. 19F is a bar chart showing observed immunoreactivity for areas corresponding to the protein DnaK.

### DETAILED DESCRIPTION OF THE INVENTION

The present invention relates to markers of Francisella tularensis infection and uses thereof. More specifically, the invention relates to markers of Francisella tularensis infection and their uses as correlates of protection.

The present invention provides a set of biomarkers for tularaemia, selected from the group consisting of: dihydrolipoamide succinyltransferase component of 2-oxoglutarate dehydrogenase complex, 50S ribosomal protein L7/L12, 30S ribosomal protein S1, DNA-directed RNA polymerase alpha subunit, Acetyl-CoA carboxylase, Outer membrane associated protein FopA, Peroxidase/catalase, Chaperone protein DnaK, Pyruvate dehydrogenase E2 component, Chaperone protein groEL, Hypothetical membrane protein, and any combination thereof.

By the term “biomarker”, also referred to as “biological marker”, it is meant a molecule that is used as an indicator of a particular state. In the context of the present application, the biomarker is an immunogenic protein, also referred to as “immunoreactive antigen” or “commonly reactive antigen”, that is reactive with sera from subjects previously infected with or vaccinated against tularaemia.

Tularemia, or “tularaemia”, refers to a spectrum of diseases caused by the Gram-negative bacterium Francisella tularensis. F. tularensis is pathogenic for many mammalian species including humans, primates, and rodents. Several subspecies exist, most notably the clinically relevant tularensis (Type A) and holarctica (Type B). Type A is associated with mortality rates of up to 60%, while Type B is associated with a less severe clinical manifestation and lower mortality rates.

The biomarkers of the present invention include dihydrolipoamide succinyltransferase component of 2-oxoglutarate dehydrogenase complex, 50S ribosomal protein L7/L12, 30S ribosomal protein S1, DNA-directed RNA polymerase alpha subunit, Acetyl-CoA carboxylase, Outer membrane associated protein, fopA, Peroxidase/catalase, Chaperone protein dnaK, Pyruvate dehydrogenase E2 component, Chaperone protein groEL, and Hypothetical membrane protein. Any combination of the aforementioned proteins is also included within the scope of the present invention. These proteins are described in Table 1.

TABLE 1
Downselected protein biomarkers of tularemia infection and vaccination. The locus
tag of the proteins refers to the Francisella tularensis SCHU S4 genome sequence.

| Locus tag | Protein Name | MW (kD) | pl | SEQ ID NO: |
|---|---|---|---|---|
| FTT_0077 | dihydrolipoamide succinyltransferase component of | 57.5 | 5.83 | 1 |
| | 2-oxoglutarate dehydrogenase complex | | | |
| FTT_0143 | 50S ribosomal protein L7/L12 | 12.8 | 4.62 | 2 |
| FTT_0183 | 30S ribosomal protein S1 | 61.5 | 5.16 | 3 |
| FTT_0350 | DNA-directed RNA polymerase alpha | 35.3 | 4.93 | 4 |
| | subunit | | | |
| FTT_0472 | Acetyl-CoA carboxylase, biotin carboxyl carrier | 16.4 | 5.00 | 5 |
| | protein subunit | | | |
| FTT0583 | Outer membrane associated protein, FopA | 41.4 | 5.58 | 6 |
| FTT_0721c | Peroxidase/catalase | 82.5 | 5.37 | 7 |
| FTT_1269c | Chaperone protein dnaK | 69.21 | 4.88 | 8 |
| FTT_1484c | Pyruvate dehydrogenase E2 component | 67.3 | 4.77 | 9 |
| FTT_1696c | Chaperone protein, groEL | 57.3 | 4.96 | 10 |
| FTT_1778c | Hypothetical membrane protein FTT_1778c | 13.7 | 8.67 | 11 |

Each of the biomarkers as described above may independently be reactive with tularaemia-exposed sera at a frequency of about 30-100%. For example, each of the biomarkers may independently be reactive with sera at a frequency of about 30, 35, 40, 45, 50, 55, 60, 65, 70, 75, 80, 85, 90, 95, or 100%, or any frequency there between.

The present invention also provides a method of evaluating immunity against tularaemia in a subject, comprising:
a. contacting serum from the subject with the biomarkers of the present invention;
b. evaluating immunoreactivity of the serum to the biomarkers;
c. determining the protection against tularaemia based on immunoreactivity to the biomarkers.

By the term “immunity”, it is meant the ability or capability to avoid infection or disease or lessen its effects. In the context of the present invention, “immunity against tularaemia” indicates that the subject is protected against tularaemia. The particular aspect of immunity being evaluated by the method of the present invention is humoral immunity. As described above, humoral immunity, including specific antibody responses has been observed following natural Francisella infections or vaccination (Dennis et al, 2001; Saslaw & Carhart, 1961; Carlsson et al, 1979; Viljanen et al, 1983). Because subjects that have recovered from Types A and B Francisella infections rarely show signs of disease following a second exposure, those subjects represent a population that is immune to further challenge. Thus, evaluating immunity of subjects against tularaemia based on antibody response may provide a direct correlation to overall immunity to tularaemia.

The subject in which the immunity against tularaemia is evaluated may be any suitable subject. The subject may be a mammal; for example, and without wishing to be limiting in any manner, the subject may be a human, a primate, a rodent (such as a rabbit or mouse), any other suitable mammalian subject. The subject may or may not have been exposed to tularaemia prior to evaluation of immunity.

The immunoreactivity of the serum to the selected biomarkers may be evaluated by any suitable method known in the art. For example, and without wishing to be limiting in any manner, two-dimensional polyacrylamide gel electrophoresis (2D PAGE) Western blotting may be used to evaluate immunoreactivity of the biomarkers to the subject's serum; once proteins are isolated they may be identified by mass spectrometry. Other suitable methods known to those of skill in the art may also be used to evaluate immunoreactivity to the biomarkers. This could include the use of a Francisella proteome microarray (Eyles et al, 2007), an ELISA screening method, a dot-blot technique, or any other technique or method relying on antibody-antigen reactivity. Persons of skill in the art would be familiar with such techniques, which have been described in detail in the art.

In the method described above, a determination of protection against tularaemia can be made based on the immunoreactivity of the subject's serum to the biomarkers; in other words, a person of skill in the art can determine whether the subject is protected against tularaemia or not. Immunoreactivity to selected biomarkers of the present invention may be an indicator that the subject is protected against tularaemia; immunoreactivity to a combination of the biomarkers of the present invention is a good indication of protection.

The present invention further provides a method of predicting vaccine efficacy, comprising:
a. contacting serum from a vaccinated model of tularaemia with the biomarkers of the present invention;
b. evaluating immunoreactivity of the serum to the biomarkers;
c. correlating the immunoreactivity to immunoprotective status of the model; and
d. predicting vaccine efficacy in human based on the correlation of step c.

By “predicting vaccine efficacy”, it is meant that the efficacy of a vaccine in conferring immunity to a human subject is predicted by the method presently described. The serum from a vaccinated model of tularaemia is obtained. The model of tularaemia may be a human or animal model; in a non-limiting example, the model of tularaemia may be an animal model such as, but not limited to a mouse model, rabbit model, primate model, or other suitable animal model that simulates a human with respect to its susceptibility or reaction to tularaemia. In one specific example, the animal model may be a mouse, rabbit, non-human primate, or human. The immunoreactivity of the serum from the model of tularaemia to the biomarkers of the present invention is then evaluated.

The immunoreactivity to the selected biomarkers may be evaluated by any method known in the art. For example, and without wishing to be limiting in any manner, two-dimensional polyacrylamide gel electrophoresis (2D PAGE) Western blotting may be used to evaluate immunoreactivity of the biomarkers to the subject's serum; once proteins are isolated they may be identified by mass spectrometry. Other suitable methods known to those of skill in the art may also be used to evaluate immunoreactivity to the biomarkers, including a Francisella proteome microarray (Eyles, et al, 2007), an ELISA screening method, a dot-blot technique, or any other technique or method relying on antibody-antigen reactivity.

The immunoreactivity of the serum from the model of tularaemia is then correlated to the immunoprotective status of the model. The immunoprotective status refers to whether the model is protected against tularaemia or not; this may be determined by any suitable method known in the art, for example, but not limited to subsequent exposure to tularaemia or challenge with an appropriate tularaemia strain.

Prediction of the vaccine efficacy in a human may be made based on the correlation between immunoreactivity and immunoprotective status as described above. The pattern of immunoreactivity in the model is observed and compared to the immunoreactivity in a vaccinated human. If the markers of the present invention show a similar pattern of immunoreactivity in the human as in the model, then the skilled person can extrapolate that the human will have the same immunoprotective status as the model. For example, and without wishing to be limiting in any manner, if the model is protected and the human's markers show the same pattern of immunoreactivity as the model's, then it may be extrapolated that the human is protected.

The present invention will be further illustrated in the following examples. However, it is to be understood that these examples are for illustrative purposes only and should not be used to limit the scope of the present invention in any manner.

### Example 1

### Sera Preparations

Four distinct collections of human sera were used in Examples 2 and 3, including human tularemia patients (two separate groups), LVS-vaccinated laboratory personnel, and clinical trial subjects immunized with LVS. The details of each sera screened are shown in Table 2 and summarized briefly below.

The Type B convalescent sera were obtained from patients diagnosed with tularemia in a region of Sweden, where the disease is considered endemic. Control sera were obtained from individuals with no history of tularemia or a tularemia-like disease. In total, sera were obtained from 12 tularemia patients and 3 healthy individuals with no history of tularemia. Since Type A strains are endemic to North America only, the Swedish patients were exclusively infected with type B strains. The route of infection for the majority of these patients was intradermal.

The Type A convalescent sera were obtained from a subset of 59 subjects with presumed or confirmed cases of tularemia reported on Martha's Vineyard between 2000 and 2006. Approximately 60% were thought to be due to inhalation of the bacterium (Matyas et al, 2007; Feldman et al, 2003). Sera from the first physician visit were available from 12 confirmed Type A tularemia patients.

Two sets of sera from separate human LVS vaccinations were obtained. The first set, in which at-risk laboratory workers in Sweden were immunized with LVS NDBR101 Lot 11, was comprised of 5 sets of paired pre- and post-vaccination samples and an additional 3 post-vaccination serum samples. NDBR lot 11 was prepared as per the vial instructions. Briefly, the vaccine preparation was reconstituted in 2.0 ml of water to give a concentration of 2.5×10⁹ CFU/ml. A droplet of approximately 20 μl (containing ˜5×10⁷ CFU) was administered by scarification using a bifurcated needle that was used to puncture the skin.

The second human vaccinee serum set was from subjects vaccinated with a recently manufactured lot of LVS (DVC lot 17) and was obtained from a Phase I clinical trial carried out at the Baylor College of Medicine, Houston, Tex. The vaccine used was manufactured at Cambrex Bio Science, Baltimore, Md., under contract with Dynport Vaccine Company LLC (DVC). Vaccine was administered as described previously (El Sahly et al, 2009). Briefly, the lyophilized vaccine was reconstituted with 0.25 ml of sterile water for injection yielding a vaccine concentration of 1.6 10⁹ CFU/ml. The study design and administration of the vaccine was described in detail previously (El Sahly et al, 2009), with dosages of 10³, 10⁵, 10⁷ and 10⁹ CFU/ml administered by scarification with a bifurcated needle. Five paired sera (pre- and 42 days post-vaccination) and three unpaired sera (post-vaccination) were provided.

TABLE 2
Summary of sera used in screening.

| | | | Date or | | | |
|---|---|---|---|---|---|---|
| | | | length of | | | |
| | | | time post | | | |
| | Sample | | infection/vaccination | | | |
| | reference | Date of | serum | | | |
| Type | number | diagnosis | drawn | Route | Pairing | Origin |
| Type B convalescent | 1651 | Unknown | 42 months | | NA | Sweden |
| Type B convalescent | 1653 | Unknown | 17 months | | NA | Sweden |
| Type B convalescent | 1657 | Unknown | 44 months | | NA | Sweden |
| Type B convalescent | 1661 | Unknown | 18 months | | NA | Sweden |
| Type B convalescent | 1663 | Unknown | 18 months | | NA | Sweden |
| Type B convalescent | 1671 | Unknown | 18 months | | NA | Sweden |
| Type B convalescent | 1673 | Unknown | 18 months | | NA | Sweden |
| Type B convalescent | 1679 | Unknown | 42 months | | NA | Sweden |
| Type B convalescent | 1683 | Unknown | 18 months | | NA | Sweden |
| Type B convalescent | 1687 | Unknown | 17 months | | NA | Sweden |
| Type B convalescent | 1691 | Unknown | 30 months | | NA | Sweden |
| Type B convalescent | 1693 | Unknown | 17 months | | NA | Sweden |
| Type A convalescent | f0703 | Unknown | 08/02/2006 | | NA | NE, USA |
| Type A convalescent | f0709 | 2005 | 22/02/2006 | | NA | NE, USA |
| Type A convalescent | f0711 | Unknown | 24/05/2006 | | NA | NE, USA |
| Type A convalescent | f0715 | Unknown | 14/03/2006 | | NA | NE, USA |
| Type A convalescent | f0722 | Unknown | 04/04/2005 | | NA | NE, USA |
| Type A convalescent | f0723 | Unknown | 04/04/2005 | | NA | NE, USA |
| Type A convalescent | f0753 | 2006 | 21/11/2007 | | NA | NE, USA |
| Type A convalescent | f0754 | 2007 | 21/11/2007 | | NA | NE, USA |
| Type A convalescent | f0756 | 2006 | 21/12/2007 | | NA | NE, USA |
| Type A convalescent | f0757 | 2006 | 21/12/2007 | | NA | NE, USA |
| Type A convalescent | f0758 | 2007 | 05/01/2008 | | NA | NE, USA |
| Type A convalescent | f0759 | 1999 | 16/01/2008 | | NA | NE, USA |
| NDBR Lot 11 Vaccinee | 201 | NA | 42 days | Scarification | NA | Sweden |
| NDBR Lot 11 Vaccinee | 202 | NA | 42 days | Scarification | NA | Sweden |
| NDBR Lot 11 Vaccinee | 203 | NA | 42 days | Scarification | NA | Sweden |
| NDBR Lot 11 Vaccinee | 204 | NA | 42 days | Scarification | NA | Sweden |
| NDBR Lot 11 Vaccinee | 205 | NA | 42 days | Scarification | NA | Sweden |
| NDBR Lot 11 Vaccinee | 206 | NA | 42 days | Scarification | NA | Sweden |
| NDBR Lot 11 Vaccinee | 207 | NA | 42 days | Scarification | NA | Sweden |
| NDBR Lot 11 Vaccinee | 208 | NA | 42 days | Scarification | NA | Sweden |
| NDBR Lot 11 Control | 111 | NA | Control | Scarification | NA | Sweden |
| NDBR Lot 11 Control | 112 | NA | Control | Scarification | NA | Sweden |
| NDBR Lot 11 Control | 113 | NA | Control | Scarification | NA | Sweden |
| NDBR Lot 11 Control | 114 | NA | Control | Scarification | NA | Sweden |
| NDBR Lot 11 Control | 115 | NA | Control | Scarification | NA | Sweden |
| DVC Lot 17 Vaccinee | 200162501 | NA | 2 | Scarification | 02855 | USA |
| | | | | (10⁹ cfu/mL) | | |
| DVC Lot 17 Vaccinee | 200161436 | NA | 10 | Scarification | 02855 | USA |
| | | | | (10⁹ cfu/mL) | | |
| DVC Lot 17 Vaccinee | 200139305 | NA | 2 | Scarification | 02816 | USA |
| | | | | (10⁵ cfu/mL) | | |
| DVC Lot 17 Vaccinee | 200162408 | NA | 10 | Scarification | 02816 | USA |
| | | | | (10⁵ cfu/mL) | | |
| DVC Lot 17 Vaccinee | 200162457 | NA | 02 | Scarification | 02858 | USA |
| | | | | (10⁹ cfu/mL) | | |
| DVC Lot 17 Vaccinee | 200161482 | NA | 10 | Scarification | 02858 | USA |
| | | | | (10⁹ cfu/mL) | | |
| DVC Lot 17 Vaccinee | 200161191 | NA | 10 | Scarification | 02840 | USA |
| | | | | (10⁷ cfu/mL) | | |
| DVC Lot 17 Vaccinee | 200162938 | NA | 10 | Scarification | 02838 | USA |
| | | | | (10⁷ cfu/mL) | | |
| DVC Lot 17 Vaccinee | 200139359 | NA | 02 | Scarification | 02807 | USA |
| | | | | (10⁵ cfu/mL) | | |
| DVC Lot 17 Vaccinee | 200162272 | NA | 10 | Scarification | 02807 | USA |
| | | | | (10⁵ cfu/mL) | | |
| DVC Lot 17 Vaccinee | 200139603 | NA | 02 | Scarification | 02832 | USA |
| | | | | (10⁷ cfu/mL) | | |
| DVC Lot 17 Vaccinee | 200162794 | NA | 10 | Scarification | 02832 | USA |
| | | | | (10⁷ cfu/mL) | | |
| DVC Lot 17 Vaccinee | 200208090 | NA | 10 | Scarification | 02875 | USA |
| | | | | (10³ cfu/mL) | | |

NA—not applicable

### Example 2

### Two-Dimensional Polyacrylamide Gel Electrophoresis Western Blotting

The four collections of human sera described in Example 1 were used in two-dimensional polyacrylamide gel electrophoresis (2D PAGE) Western blotting experiments in order to determine the repertoire of immunoreactive proteins for each serum sample. Briefly, the proteins of a bacterial cell lysate were separated in two dimensions—by protein isoelectric point then by protein molecular mass using 2D-PAGE, as described in Twine et al (2005; 2010). Resolved proteins were then transferred to nitrocellullose membrane by electroblotting and the membrane was subsequently incubated with serum from Example 1. Antibodies in the serum recognised their cognate antigen on the membrane and this antibody binding was subsequently detected, generating a pattern of immunoreactive proteins.

Francisella tularensis Δwbtl, a mutant strain lacking the O-antigen, was used as the protein antigen in blotting experiments. Briefly, bacteria were grown in modified Cysteine Heart agar (CHA) for 24-36 h at 37° C. within a BioSafety (BS) Level 3 containment facility. Plate grown bacteria were harvested directly into lysis buffer (5 M Urea, 2M Thiourea, 4% CHAPS, 0.5% ASB-14), as described in earlier work (Twine et al, 2005), in order to solublise bacterial proteins. A portion (10%) of all cell lysates were plated on CHA and checked for sterility after incubation at 37° C. for 36 hours before release of bacteria from the BS Level 3 facility. The resulting proteins in the lysate were quantified using a Bradford protein assay.

The lysates were separated using two-dimensional polyacrylamide gel electrophoresis (2D PAGE) Western blotting. Briefly, proteins were separated in the first dimension by isoelectric point, with 100 ug of bacterial proteins, dissolved in Immobilised pH gradient solutions (Biolytes (3-10): Biolytes stock (3-10, Biorad) and 10 μg Orange G). Immobilised pH gradient (IPG) strips were rehydrated with bacterial proteins, as described in the manufacturer's instructions (Biorad, Hercules, Calif.). Isoelectric focussing was carried out using a Protean Cell (Biorad, Hercules, Calif.) with the following 24 hour programme: 200 V for 1 hour, 500 V for 1 hour, ramp to 5000 V over 5 hours, focus to 80,000 Vh at 5000 V, at 20° C., for a cumulative total of 95,000 Vh. Previous work with murine sera showed no proteins with a pI<4 or >7 reactive with sera from LVS immunized BALB/c or C57/BL6 mice (Twine et al, 2010; Twine et al, 2006); therefore, the present analyses were confined to pH 4-7. Second dimension separations were carried out using 10% SDS-PAGE. IPG strips were equilibrated with 2 mL DTT solution (0.05 g DTT, 0.1 g SDS, 0.68 mL 1M Tris HCl, pH 8.8, 3.6 g urea, 3 g glycerol, in MQ water up to 5 mL), at room temperature for 20 minutes, followed by equilibration with 2 mL iodoacetamide solution (0.2 g iodoacetamide, 0.1 g SDS, 0.68 mL 1M Tris HCl, pH 8.8, 3.6 g urea, 3 g glycerol, in 5 mL), at room temperature for 20 minutes.

Immunoblotting was carried out according to previously published methods (Mansfield, 1995; Twine et al, 2010). The human sera described in Example 1 were used at a dilution of 1:500, and secondary antibody (horseradish peroxidase labelled) was used as per the manufacturers instructions. Immunoreactivity on nitrocellulose membranes was visualised using a commercially available ECL (GE Healthcare, Baie d'Urfe, Canada). Blots were developed by 30 s or 1 minute exposure to Kodak Biomax Scientific imaging film. Developed blots were aligned with either images of the protein-stained nitrocellulose membrane, after protein transfer, or protein stained 2D-PAGE. Protein spots observed to be immunoreactive were identified as described in Example 3.

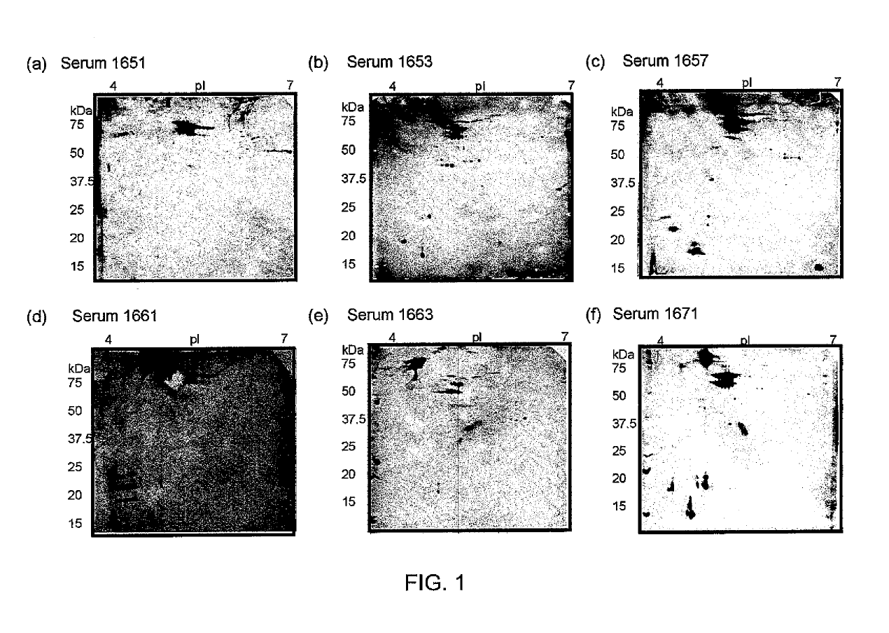

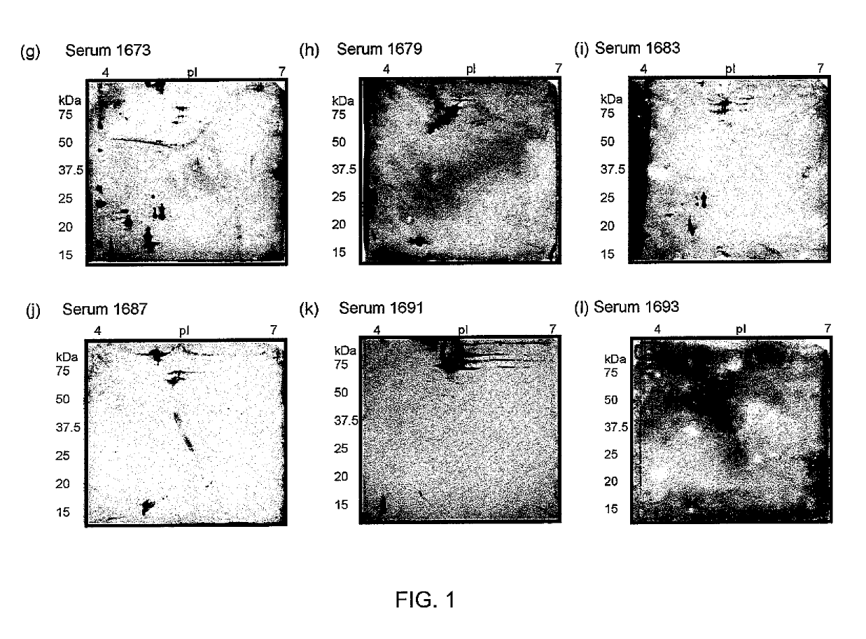

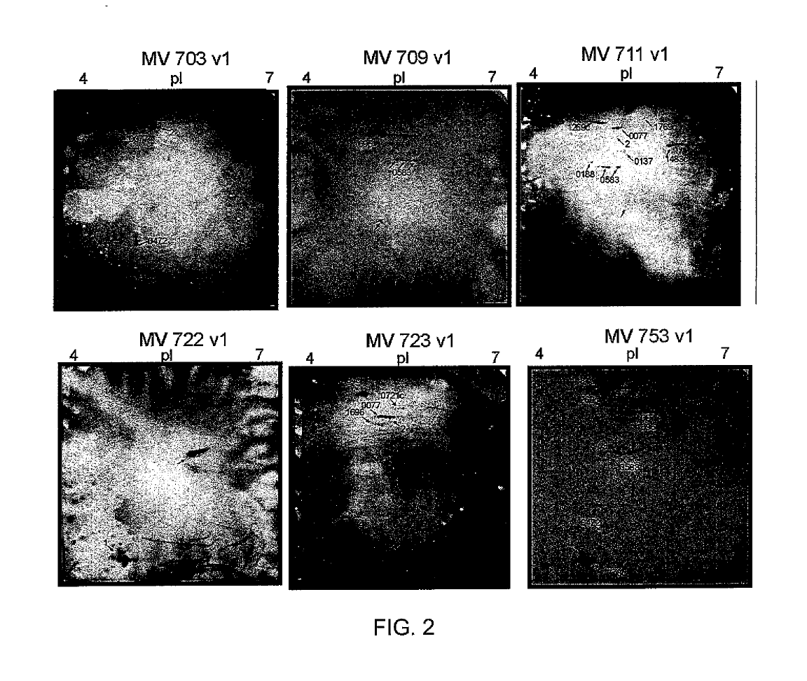

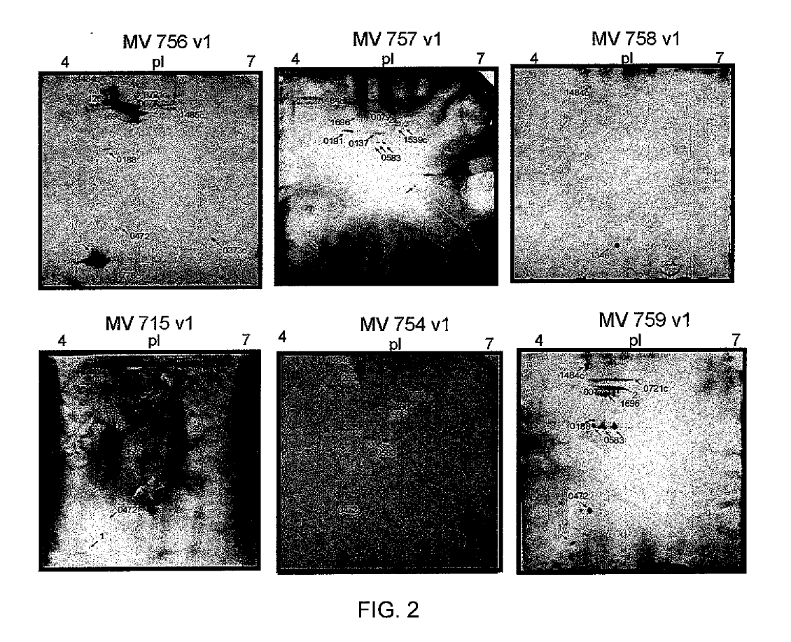

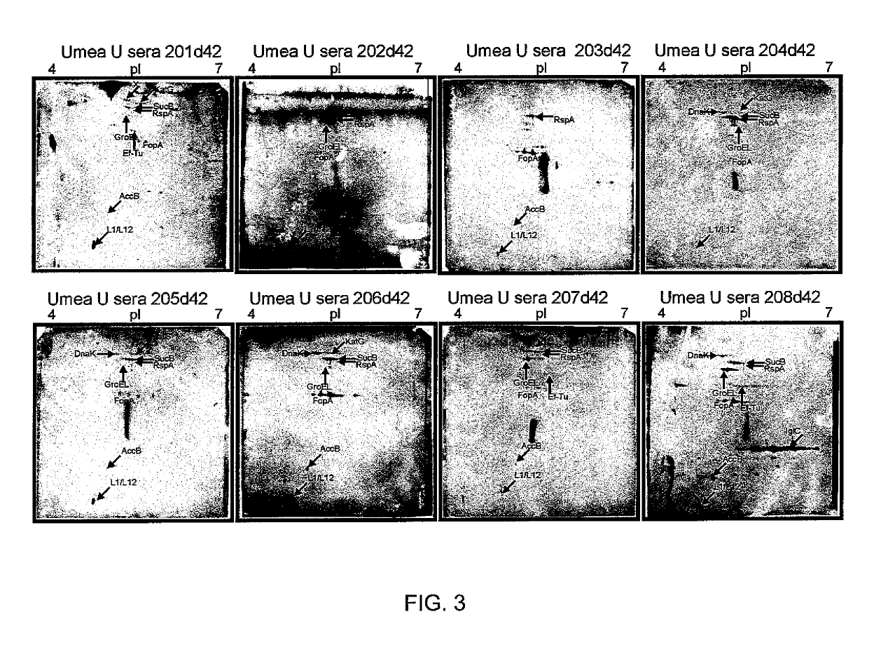

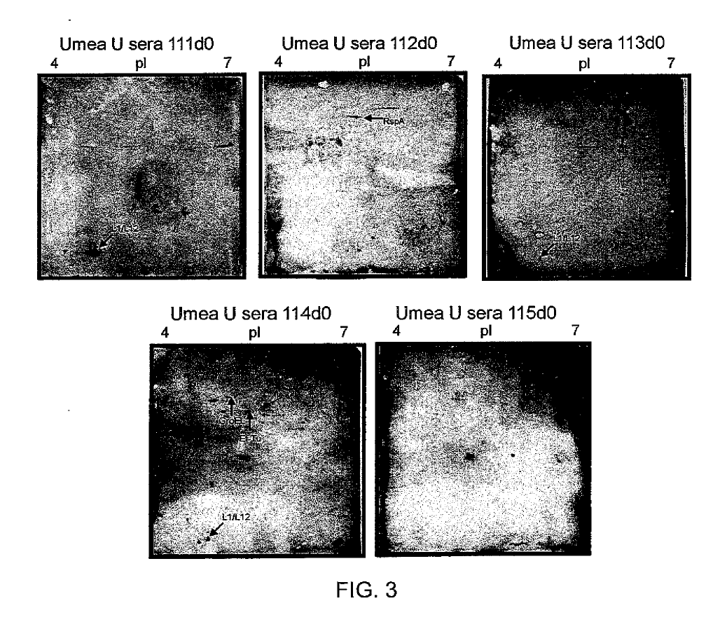

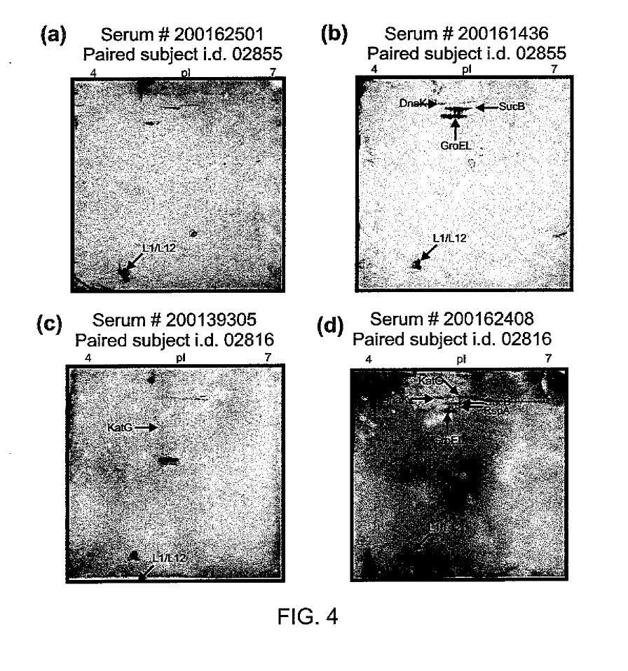

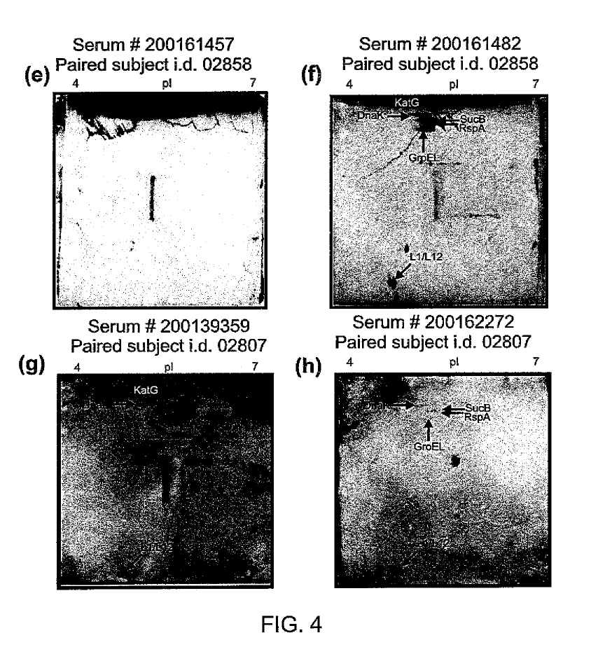

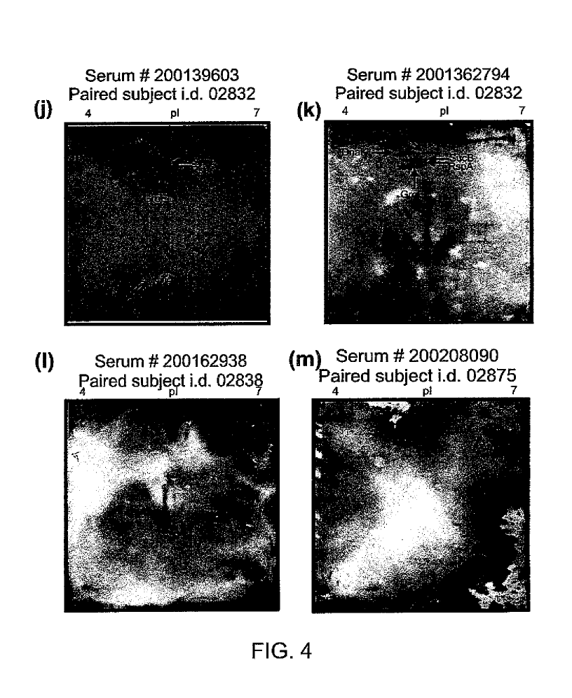

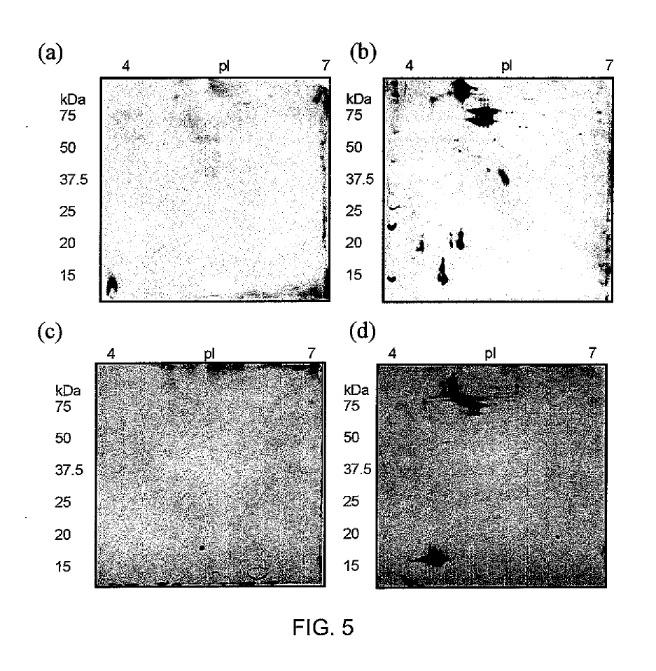

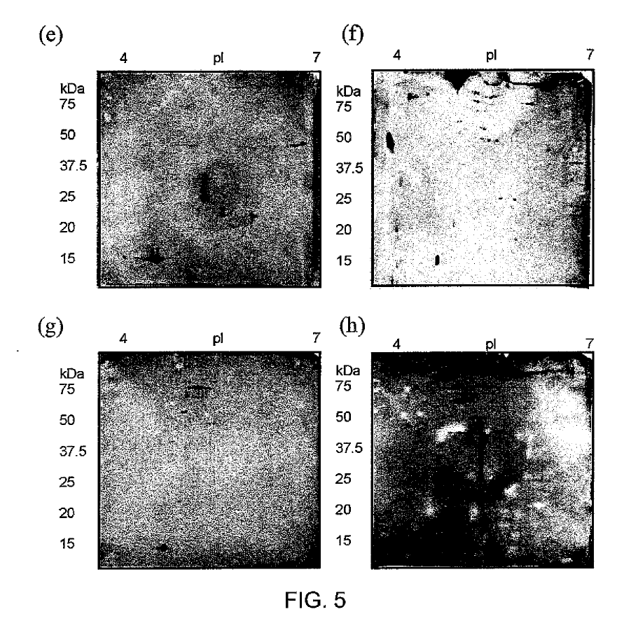

The complete series of Western blots are shown in FIGS. 1 to 4. Specifically, FIG. 1 shows Western blots probed with sera from Type B patients; FIG. 2 shows Western blots probed with sera from Type A patients; FIG. 3 shows Western blots probed with sera from LVS vaccinees (Umea); and FIG. 4 shows Western blots probed with sera from LVS vaccinees (Baylor). FIG. 5 is a comparison of representative 2D Western blots of FIGS. 1 to 4.

### Example 3

### Identification of Immunoreactive Proteins

Immunoreactive proteins observed by 2D Western blotting in Example 2 were identified by tryptic digest and mass spectrometry.

Protein spots corresponding to areas of immunoreactivity on Western blots were excised from equivalent protein stained 2D-PAGE gels and tryptically digested manually, as described previously (Twine et al, 2010; Twine et al, 2006). The in-gel digests were analyzed by nano-liquid chromatography-MS/MS as described previously (Twine et al, 2010). The peak list files of MS² spectra of the excised protein spots were searched against the translated SCHU S4 genome sequence using the MASCOT™ search engine (version 2.2.03) (Matrix Science, London, UK) for protein identification, as described in earlier work (Twine et al, 2010).

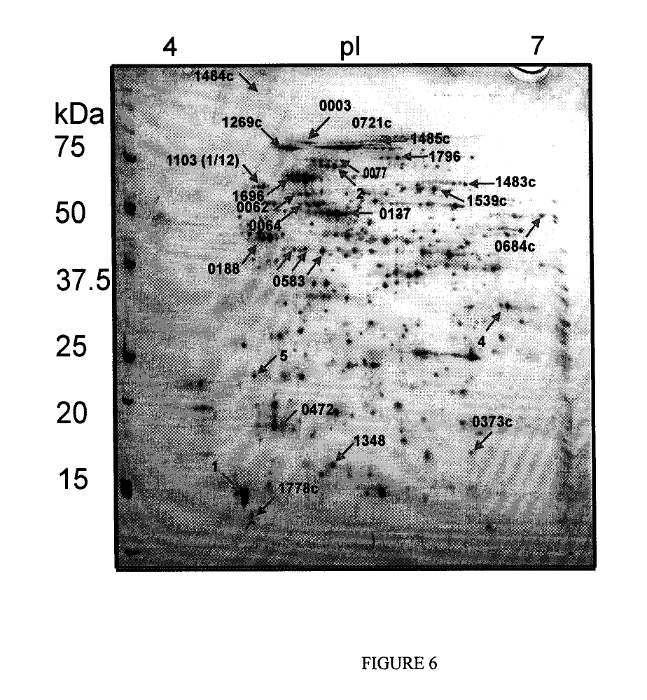

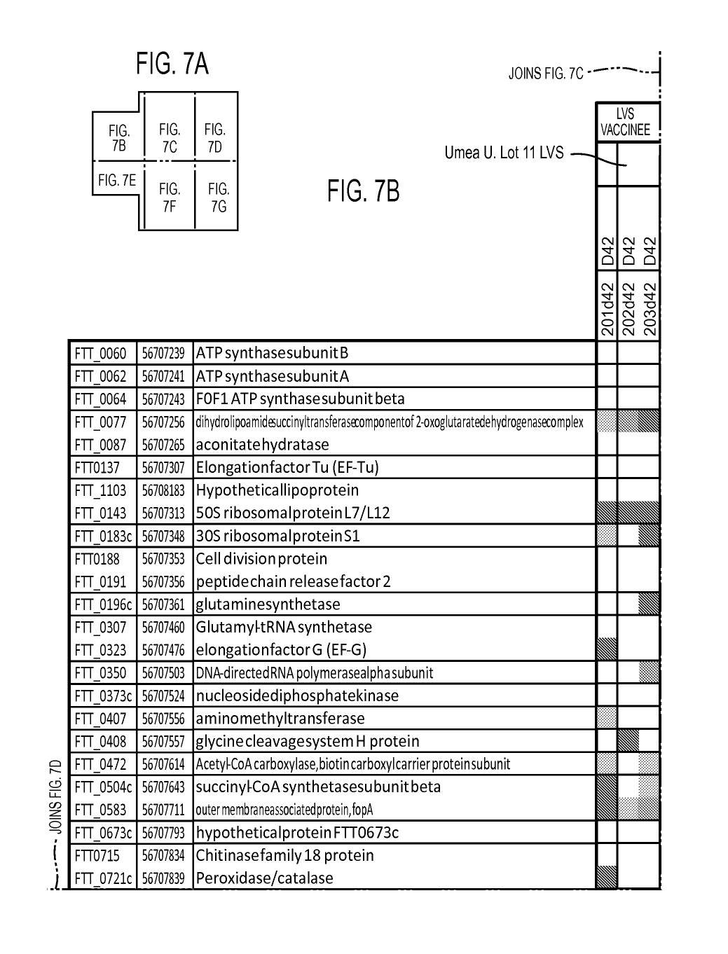

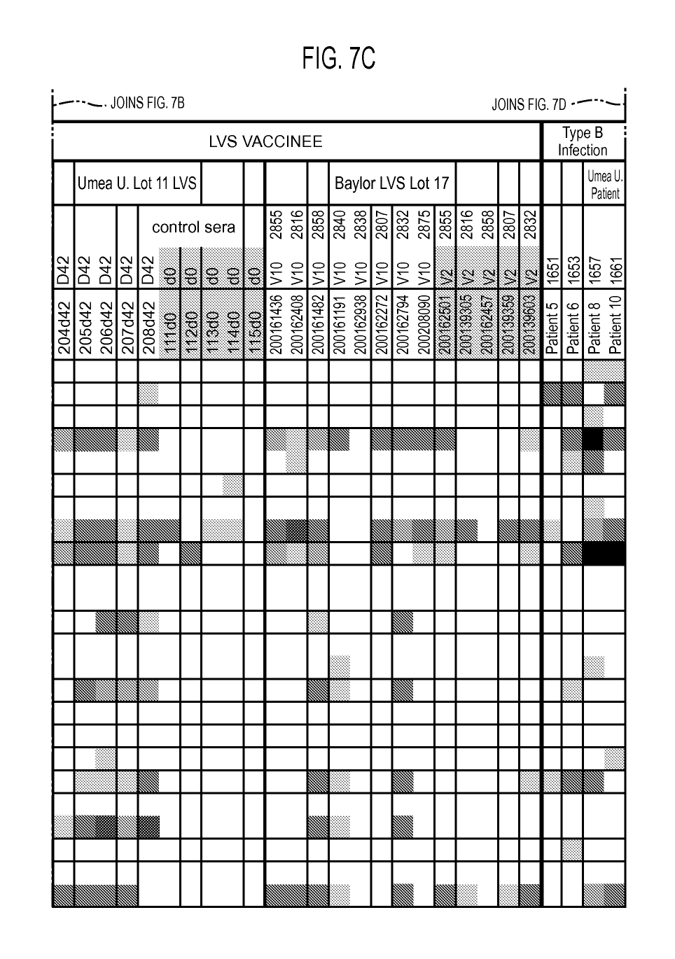

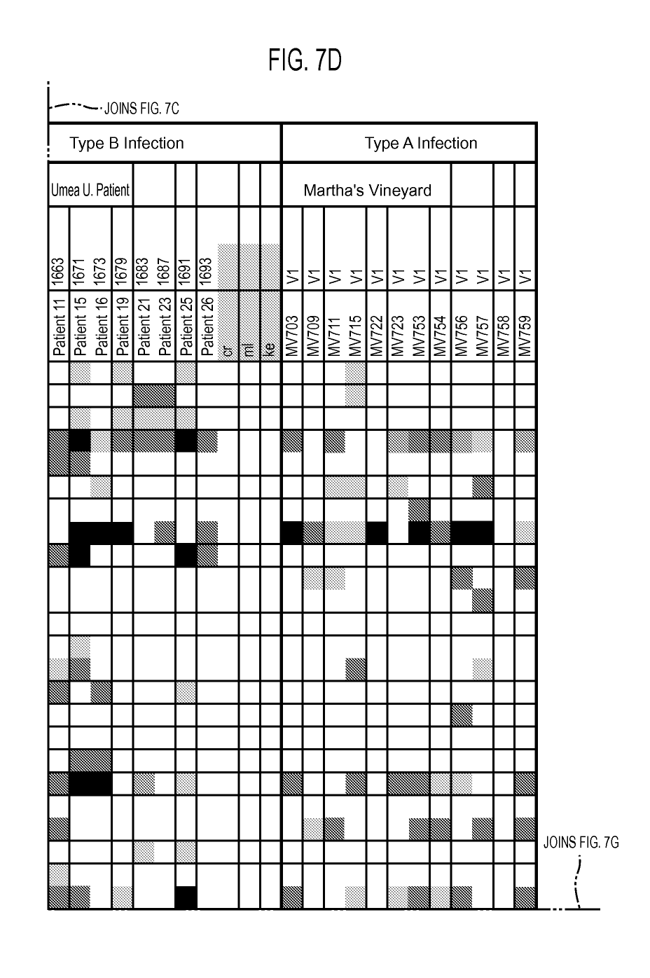

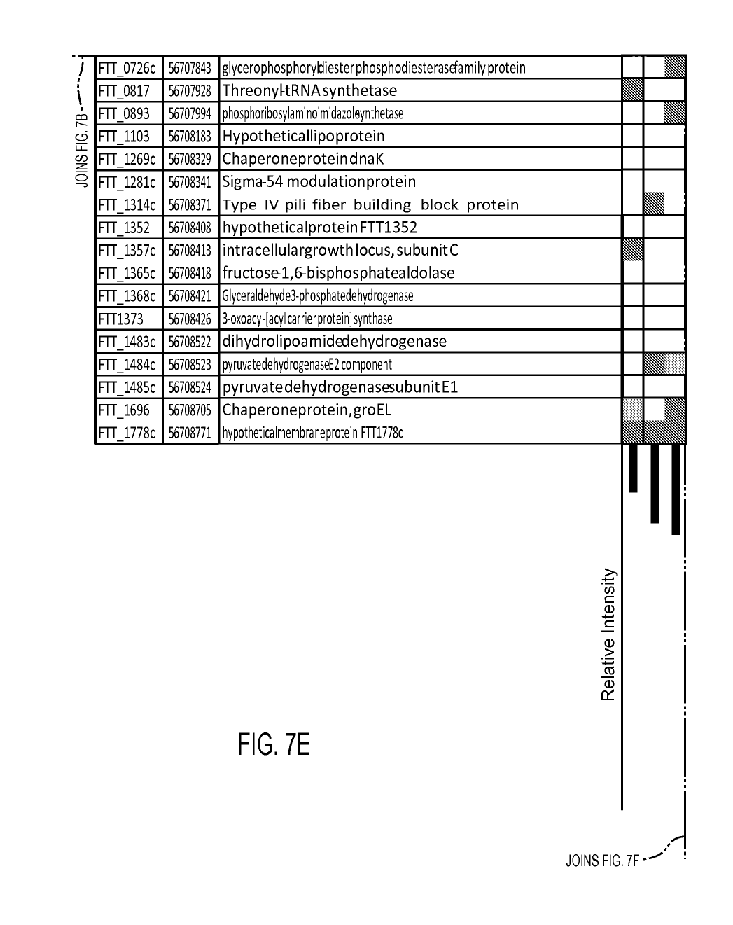

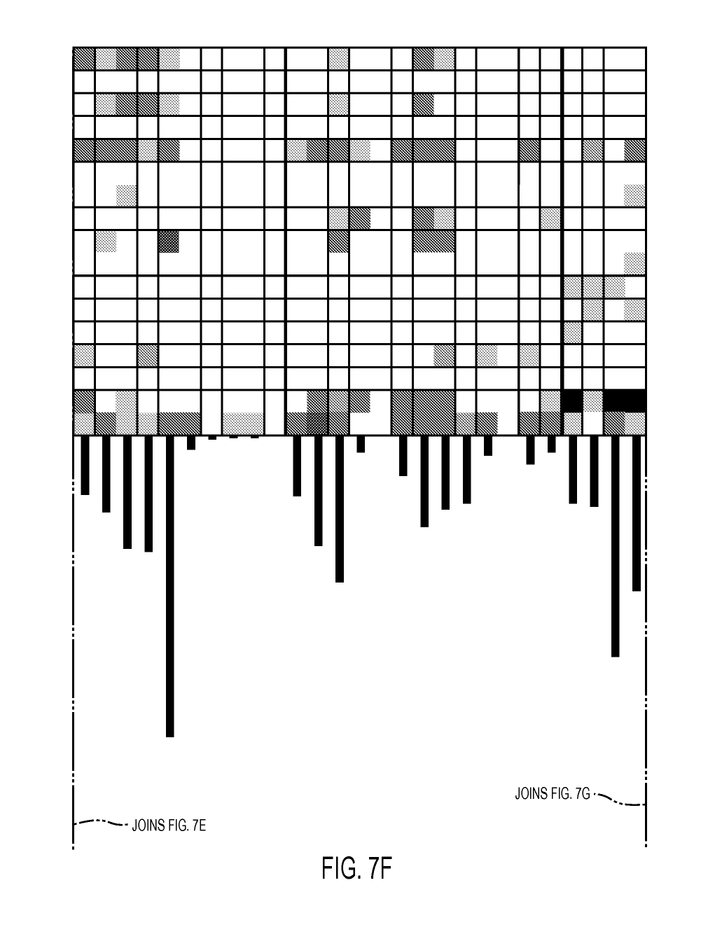

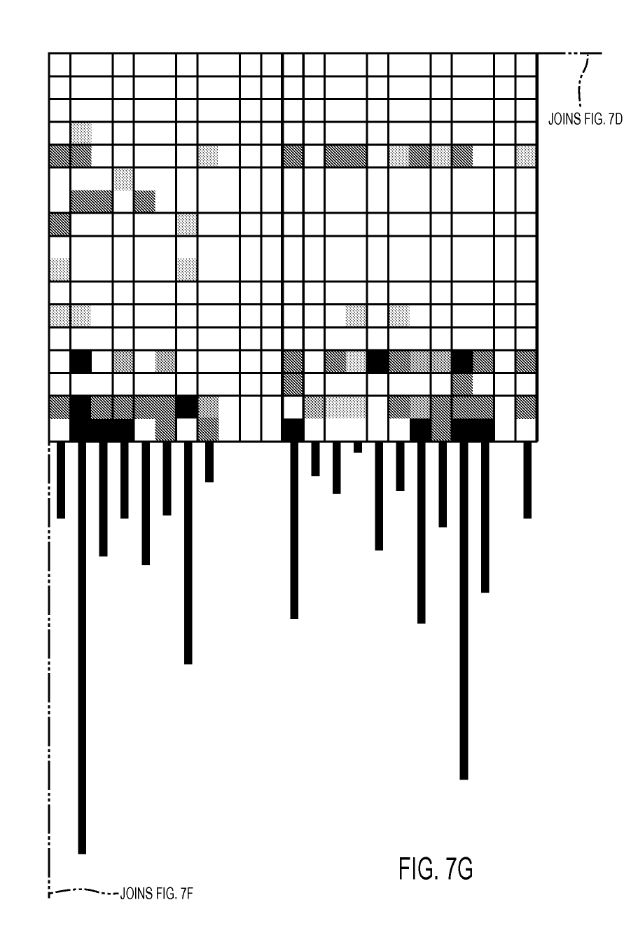

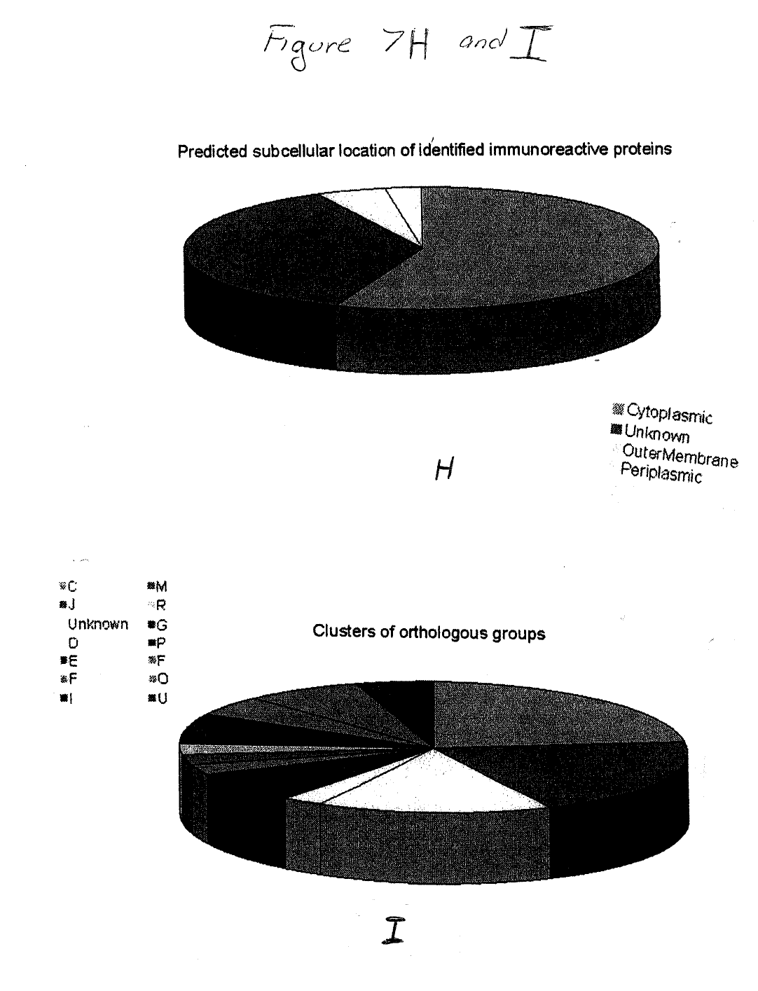

A total of 31 immunoreactive proteins were identified from Type B patients (Table 3, FIG. 6). A single protein, Chaperonin GroEL (FTT_{—}1696), was immunoreactive with all sera from all 12 patients, but with none of the control sera. The protein dihydrolipoamide succinyltransferase component of 2-oxoglutarate dehydrogenase complex (FTT_{—}0077) was immunoreactive with 11 of the 12 patient sera screened but none of the control sera. Further to this, the proteins 50S ribosomal protein L1/L12 (FTT_{—}0143), hypothetical membrane protein (FTT_{—}1778c), and acetyl CoA carboxylase (FTT_{—}0472) was immunoreactive with 8 or more patient sera screened. The proteins FTT_{—}1778c and FTT_{—}0143 focused to discrete spots on 2D-PAGE within close proximity of one another. Where immunoreactivity of these proteins was intense, it was not always possible to discern which individual protein was immunoreactive, or to measure the intensity of immunoreactivity of the individual proteins. In these cases, it was indicated that both proteins were immunoreactive and the overall intensity of immunoreactivity reported. These data are also represented visually as a matrix of immunoreactive proteins, in FIG. 7A. The shading indicates the comparative intensity of the observed immunoreactivity for each spot, as measured by densitometry, with darker shading denoting more intense immunoreactivity.

TABLE 3
A summary of proteins reactive with sera from human tularemia patient (Type A and
B) or LVS vaccinees

| | | | Study | | | | |
|---|---|---|---|---|---|---|---|
| | | | Vaccine | Vaccine | | | |
| | | | NDBR lot | DVC lot | | | |
| | GenInfo | | 11 LVS | 17 LVS | Type B | Type A | Previously |
| Locus tag | Identifier | Protein Name | (n = 8) | (n = 8) | (n = 12) | (n = 12) | reported |
| FTT_0060 | 56707239 | ATP synthase subunit B | — | — | 5 | 1 | (1), (2) |
| FTT_0062 | 56707241 | ATP synthase subunit A | 1 | — | 5 | 1 | (2), (3), (4) |
| FTT_0064 | 56707243 | F0F1 ATP synthase | — | — | 6 | — | (3), (5) |
| | | subunit beta | | | | | |
| FTT_0077 | 56707256 | Dihydrolipoamide | 8 | 7 | 11 | 8 | (1), (2), (3), (4), |
| | | succinyltransferase | | | | | (6)(7) |
| | | component of 2- | | | | | |
| | | oxoglutarate | | | | | |
| | | dehydrogenase complex | | | | | |
| FTT_0087 | 56707265 | Aconitate hydratase | — | 1 | 4 | — | (3), (6), (7) |
| FTT_0137 | 56707307 | Elongation factor Tu (EF- | | | 1 | 4 | (2), (3), (4), (6), |
| | | Tu) | | | | | (8) |
| FTT_1486c | 56708525 | Hypothetical lipoprotein | — | — | 1 | 1 | |
| FTT_0143 | 56707313 | 50S ribosomal protein | 8 | 6 | 8 | 10 | (2), (4), (6), (8) |
| | | L7/L12 | | | | | |
| FTT_0183c | 56707348 | 30S ribosomal protein S1 | 7 | 5 | 7 | — | (2), (3),(4) |
| FTT_0188 | 56603845 | Cell division protein | — | — | 1 | 4 | (2), (3), (4), (6) |
| FTT_0191 | 56707356 | Peptide chain release | | — | — | 1 | |
| | | factor 2 | | | | | |
| FTT_0196c | 56707361 | Glutamine synthetase | 4 | 2 | — | — | (1), (2) |
| FTT_0307 | 56707460 | Glutamyl-tRNA | | | 1 | — | |
| | | synthetase | | | | | |
| FTT_0323 | 56707476 | Elongation factor G (EF- | 1 | 1 | 3 | 2 | (2), (3), (4) |
| | | G) | | | | | |
| FTT_0350 | 56707503 | DNA-directed RNA | 5 | 3 | 4 | — | , (2), (3) |
| | | polymerase alpha subunit | | | | | |
| FTT_0373c | 56707524 | Nucleoside diphosphate | — | — | — | 1 | (2), (8) |
| | | kinase | | | | | |
| FTT_0407 | 56707556 | Aminomethyltransferase | 1 | — | — | — | (2) |
| FTT_0408 | 56707557 | Glycine cleavage system | 2 | — | 3 | — | |
| | | H protein | | | | | |
| FTT_0472 | 56707614 | Acetyl-CoA carboxylase, | 6 | 3 | 8 | 7 | (1), (3), (4), (8) |
| | | biotin carboxyl carrier | | | | | |
| | | protein subunit | | | | | |
| FTT_0504c | 56707643 | Succinyl-CoA synthetase | 2 | — | — | — | (7) |
| | | subunit beta | | | | | |
| FTT_0583 | 56707711 | Outer membrane | 8 | 3 | 1 | 6 | (1), (3), (4), (6) |
| | | associated protein, fopA | | | | | |
| FTT_0673c | 56708418 | Hypothetical protein | — | — | 3 | — | |
| | | FTT0673c | | | | | |
| FTT_0715 | 56707834 | Chitinase family 18 | — | — | 1 | — | (2), (3), (4), |
| | | protein | | | | | |
| FTT_0721c | 56707839 | Peroxidase/catalase | 5 | 5 | 6 | 7 | (2), (3), (4), |
| | | | | | | | (5), (6), (7), (8) |
| FTT_0726c | 56707843 | Glycerophosphoryl diester | 6 | 3 | — | — | (6) |
| | | phosphodiesterase family | | | | | |
| | | protein | | | | | |
| FTT_0817 | 56707928 | Threonyl-tRNA synthetase | 1 | — | — | — | (2), (4) |
| FTT_0893 | 56707994 | Phosphoribosylaminoimidazole | 5 | 2 | — | — | |
| | | synthetase | | | | | |
| FTT_1103 | 56708183 | Hypothetical lipoprotein | — | — | 1 | — | (1), |
| | | | | | | | (2), (3), (4), (5), |
| | | | | | | | (6) |
| FTT_1269c | 56708329 | Chaperone protein dnaK | 5 | 7 | 5 | 8 | (1), |
| | | | | | | | (2), (3), (4), (5), |
| | | | | | | | (6), (7), (8) |
| FTT_1281c | 56707258 | Sigma-54 modulation | — | — | 1 | — | (2), (8) |
| | | protein | | | | | |
| FTT_1314c | 56708371 | Type IV pili fiber building | 2 | — | 4 | — | (1), (2) |
| | | block protein | | | | | |
| FTT_1352 | 56708408 | Hypothetical protein | — | 4 | 2 | — | |
| | | FTT1352 | | | | | |
| FTT_1357c | 56708413 | Intracellular growth locus, | 3 | 3 | — | — | (2), (5) |
| | | subunit C | | | | | |
| FTT_1365c | 56708418 | Fructose-bisphosphate | — | — | 3 | — | |
| | | aldolase | | | | | |
| FTT_1368c | 56708421 | Glyceraldehyde-3- | — | — | 3 | — | (2), (8) |
| | | phosphate | | | | | |
| | | dehydrogenase | | | | | |
| FTT_1373 | 56708426 | 3-oxoacyl-[acyl carrier | — | — | 4 | 2 | (3) |
| | | protein] synthase III | | | | | |
| FTT_1483c | 56708522 | Dihydrolipoamide | — | — | 1 | — | (6) |
| | | dehydrogenase | | | | | |
| FTT_1484c | 56605015 | Pyruvate dehydrogenase, | 4 | 3 | 3 | 10 | (6), (7) |
| | | E2 component | | | | | (1), (3) |
| FTT_1485c | 56708522 | Pyruvate dehydrogenase | — | — | — | 2 | (7) |
| | | subunit E1 | | | | | |
| FTT_1696 | 56708705 | Chaperone protein, groEL | 4 | 6 | 12 | 9 | (1), (3), (4), |
| | | | | | | | (5), (6) |
| FTT_1778c | 56708771 | Hypothetical membrane | 8 | 6 | 8 | 5 | (1), (3), (4), |
| | | protein FTT1778c | | | | | |

(1) Eyles et al, 2007;
(2) Titball et al, 2007;
(3) Twine et al, 2010;
(4) Twine et al, 2006;
(5) Huntley et al, 2007;
(6) Janovska et al, 2007a;
(7) Janovska et al, 2007b;
(8) Havlasova et al, 2005)

A total of 19 proteins were identified as immunoreactive with sera from one or more of the Type A tularemia patients. No single protein was observed to be immunoreactive with all Type A tularemia patient sera screened, although the proteins pyruvate dehydrogenase E2 (FTT_{—}1484c) and ribosomal protein L7/L12 (FTT_{—}0143) were observed to be reactive with 10 of the 12 sera studied. In addition, the proteins dihydrolipoamide succinyltransferase component of 2-oxoglutarate dehydrogenase (FTT_{—}0077) and chaperonin protein DnaK (FTT_{—}1269c) were reactive with 8 of the total 12 sera.

Across all eight post-vaccination serum samples from LVS vaccinated Swedish laboratory workers (LVS NDBR101 Lot 11) screened, a total of 22 immunoreactive proteins were identified (Table 3, FIG. 7). Four proteins were observed to be immunoreactive to some degree with all of the post-vaccination sera: dihydrolipoamide succinyltransferase component of 2-oxoglutarate dehydrogenase complex (FTT_{—}0077), 50S ribosomal protein L7/L12 (FTT_{—}0143), outer membrane protein FopA (FTT_{—}0583) and hypothetical protein (FTT_{—}1778c). Of the pre-vaccination sera, one individual showed no detectable immunoreactivity. The remaining four sera showed weak immunoreactivity with the proteins 30S ribosomal protein S1 (FTT_{—}0183c), 50S ribosomal protein L7/L12 (FTT_{—}0143) and hypothetical protein (FTT_{—}1778c).

Blots probed with sera from humans vaccinated with the new CGMP formulation of LVS (DVC lot 17) showed immunoreactivity with a total of 18 proteins (Table 3). For the post-vaccination sera, no single protein was reactive with all sera; however, the proteins dihydrolipoamide succinyltransferase component of 2-oxoglutarate dehydrogenase complex (FTT_{—}0077) and chaperonin dnaK (FTT_{—}1269c) were reactive with seven of the eight post-LVS vaccination sera. It is also interesting to note that the outer membrane protein FopA (FTT_{—}0583), was reactive with only three post-vaccination sera. The pre-vaccination sera showed no reactivity (one subject) or weak reactivity towards the proteins dihydrolipoamide succinyltransferase component of 2-oxoglutarate dehydrogenase complex (FTT_{—}0077), 30S ribosomal protein S1 (FTT_{—}0183c), catalase (FTT_{—}0721c), 50S ribosomal protein L7/L12 (FTT_{—}0143), and hypothetical protein (FTT_{—}1778c). The escalating vaccine dose did not influence the repertoire of immunoreactive proteins, with the exception of post-vaccination sera from one subject (#200162938), vaccinated with 10⁷ CFU, showed no detectable immunoreactivity

FIG. 7 shows the repertoire of immunoreactive proteins catalogued for each serum sample screened. This figure also illustrates the relative intensity of each observed immunoreactive spot and shows the marked heterogeneity in the immunoreactivity. The bar chart below the matrix of immunoreactive proteins in FIG. 7A shows the sum of the comparative intensity values for the identified immunoreactive proteins. With the exception of the NDBR lot 11 LVS vaccinees, the mean total relative intensity in identified immunoreactive spots was similar for each group of sera screened. One subject from each of NDBR lot 11 LVS vaccinees, Type A and Type B tularemia patients, showed a markedly higher total intensity of immunoreactive proteins. In contrast, two other sera drawn 42 days post-vaccination with 10⁵ CFU DVC lot 17 LVS, also showed comparatively low immunoreactivity. The greatest total relative intensity of identified immunoreactive proteins was observed for the vaccination dose of 10⁹ CFU

The properties of the reactive proteins were examined according to computationally predicted features to determine whether a particular type of protein was over-represented. First, the PSORT1b algorithm was used, an algorithm that predicts the subcellular localization of proteins based upon amino acid sequences (Gardy et al, 2005). FIG. 7B shows graphically that the vast majority of identified immunoreactive proteins were predicted to be cytoplasmic in location (56%), with 36% of the proteins of unknown location (36%). The remaining proteins were localized to various locations, including the outer membrane and periplasm. Secondly, the identified proteins were classified according to the Clusters of Orthologous Groups (http://www.ncbi.nlm.nih.gov/COG/; Tatusov et al, 1997; Tatusov et al, 2003) and the identified proteins were classified according to predicted function. FIG. 7C shows 23% of the identified proteins were predicted to be involved in energy production and conversion (COG C), 20% predicted to be involved in translation, and 15% to be of unknown function

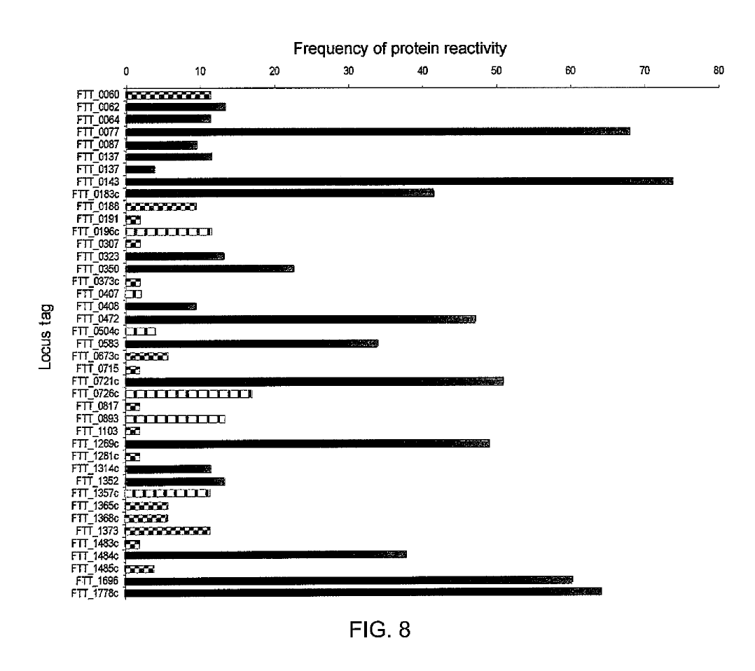

FIG. 8 shows a graphical representation of the frequency with which each immunoreactive protein was identified, regardless of experimental group. Of note, the protein dihydrolipoamide succinyltransferase component of 2-oxoglutarate dehydrogenase complex (FTT_{—}0077) was reactive with 70% of all sera screened. By contrast, the outer membrane protein FopA was observed to be reactive with sera from all LVS NDBR lot 11 vaccinees, but less than half of the subjects from other groups. From this graph, and the matrix of immunoreactive proteins in FIG. 7A, 11 proteins were identified as commonly reactive antigens, reactive with both patient and vaccinee sera, with a minimum frequency of 30%. These included dihydrolipoamide succinyltransferase component of 2-oxoglutarate dehydrogenase complex (FT_{—}0077), 50S ribosomal protein L7/L12 (FTT_{—}0143), 30S ribosomal protein S1 (FTT_{—}0183), DNA-directed RNA polymerase alpha subunit (FTT_{—}0350), Acetyl-CoA carboxylase (FTT0472), Outer membrane associated protein FopA (FTT_{—}0583), Peroxidase/catalase FTT_{—}0721c), Chaperone protein DnaK (FTT_{—}1269c), Pyruvate dehydrogenase E2 component (FTT_{—}1484c), Chaperone protein groEL (FTT1696), Hypothetical membrane protein (FTT_{—}1778c).

It was observed that the repertoire of proteins reactive with sera from individuals recovering from natural Type A and B infection showed a great deal of overlap, as shown in FIG. 7. This is interesting, given that the most common route of infection for Type A Francisella in the Martha's Vineyard patients is by inhalation (Matyas et al, 2007), whereas the most common route of infection in the European Type B-infected group is presumed to be arthropod-borne intradermal (Sjostedt, 2007). Therefore, the antibody repertoire generated in response to natural infection with Type A or B strains of Francisella has a large degree of similarity, despite differences in bacterial strains and routes of infection. Subtle differences in the immunoproteomic profiles when screened against the same Francisella antigen, however, were also observed. For example, 80% of Type A tularemia convalescent sera showed some degree of reactivity towards the protein pyruvate dehydrogenase E2 component. By contrast, sera from Type B tularemia patients showed reactivity towards the same protein in less than 50% of patients analysed. In addition, sera from Type B tularemia patients showed reactivity with a greater repertoire of proteins, with a total of thirty-one proteins observed to be reactive with sera from Type B patients, compared with eighteen with sera from Type A patients. It is difficult, however, to draw conclusions regarding the significance of these observations, given that the exact date and route of infection for each patient is unknown, as is the longevity of the circulating anti-Francisella antibodies (a majority of patients lacked demonstrable antibody titres 25 years after infection; Ericsson et al, 1994). In relation to this, a recent study reported the repertoire of immunoreactive proteins in the sera of a laboratory worker, accidentally infected with Type A Francisella, did not markedly change over a period of 16 years (Janovska, 2007b). The one exception was a single immunoreactive protein that was not observed to be reactive at later time points after infection. Of note, 5 of the 10 identified immunoreactive proteins described here were also shown in an earlier study to be reactive with sera from patients recovering from Type A infections (Janovska, 2007b).

The reactive antigens were not evenly distributed across the proteome. For example, in terms of predicted subcellular location, cytoplasmic proteins were by far over-represented. Without wishing to be bound by theory, this may result from a bias introduced by the gel-based immunoproteomics approach, which is known to have limited capability to resolve very large, small or hydrophobic proteins. A recently developed alternative is the proteome chip, where cell-free expressed proteins immobilized on microarray style chips are probed with immune sera (Sundaresh et al, 2007; Eyles et al, 2007). This approach was used to screen sera from LVS vaccinated mice and did not show the same bias towards cytoplasmic proteins. However, this approach also has limitations, including potential improper protein folding and lack of post-translational modifications of many proteins (Felgner et al, 2009). For many laboratories, the cost of this proteome chip approach can also be prohibitive. Aside from possible limitations of any experimental approach, and without wishing to be bound by theory, it may also be that certain functional categories of proteins are selectively recognized by the human immune system. The characteristics of the identified antigenic proteins that allow them to be selectively recognized by the immune system have not been identified.

No single protein was seen to be immunoreactive with all sera screened. However, eleven proteins were observed to react with the majority of sera screened and are denoted ‘commonly reactive proteins’. Together, reactivity with a combination of these proteins may be predictive of vaccinees' protection from challenge with virulent strains. Since the incidence of re-infection is extremely low, patients recovering from either Type A or B tularemia may be assumed to be protected against re-infection. Thus, antigens common to patient and vaccinee sera are more likely to serve as potential correlates of protection. Therefore, the immunoreactive proteins identified herein may be used to correlate with the protective status of the host.

Due to the inability to conduct vaccine efficacy studies in humans, development and evaluation of vaccine candidates will rely on animal models, bridging efficacy to humans based on correlates of protection. Several immunoproteomics studies of the murine humoral response to LVS vaccination have been reported (Eyles et al, 2007) and eleven of the present antigens have been reported previously in mice (Table 3). As other animal models of tularemia are developed and characterized, there exist opportunities to directly correlate the profile of immunoreactive proteins generated by LVS vaccination with the protective status of the host animal.

In addition to increasing understanding of the humoral immune response to primary pneumonic tularemia, systemic tularemia and tularemia vaccination, the identified immunoreactive proteins may be used to design and develop protein subunit based tularemia vaccine candidates

### Example 4

### Immunoproteomics of Non-Human Primate Model of Tularemia

An important bridge between small animal models of tularemia and humans is the characterization of tularemia in non-human primates. The non-human primate (NHP) model of tularemia used in this Example was developed by Battelle Biomedical Research Centre (BBRC) in Rhesus macaques.

A large scale LVS vaccination of non-human primate was carried out by BBRC (Battelle Study No. 985-G006023: Francisella tularensis LVS Vaccination Efficacy Against a F. tularensis SCHU S4 Aerosol Exposure in Rhesus Macaques). This was a large scale LVS efficacy study comprised 40 Rhesus Macaques. As shown in Table 4, six groups of animals were vaccinated with either saline or LVS, either subcutaneously (SC) or by scarification. The vaccinating dose of LVS was 8×10⁷ cfu. Groups 1, 3 and 4 were then challenged on day 35 post vaccination with 2.5×10⁶ cfu of SCHU S4 by aerosol. Animals in groups 2, 5 and 6 were challenged with the same dose of SCHU S4 by aerosol on day 63 post vaccination. Sera collected from animals pre-vaccination and prior to challenge, prior to challenge, were screened by 2D Western blotting (see Example 2).

TABLE 4
Non-human primate LVS vaccination
efficacy study design and mortality data. In each
case, challenge dose was 2.5 × 10⁶ cfu of SCHU S4 by aerosol.

| | | | | Challenge | |
|---|---|---|---|---|---|
| | NHP | | | day | Number |
| | per | Vaccine | Vaccination | (post | of |
| Group | group | dose | route | vaccination) | survivors |
| 1 | 4 | Saline | 2 by SC | 35 | 0 |
| | | | 2 by Scar | | |
| 2 | 4 | Saline | 2 by SC | 63 | 0 |
| | | | 2 by Scar | | |
| 3 | 10 | 8 × 10⁷ cfu | Scar | 35 | 7 |
| 4 | 10 | 8 × 10⁷ cfu | SC | 35 | 8 |
| 5 | 10 | 8 × 10⁷ cfu | Scar | 63 | 8 |
| 6 | 10 | 8 × 10⁷ cfu | SC | 63 | 7 |

In addition, Table 4 indicates the proportion of animals that survived challenge, with none of the saline-vaccinated animals surviving. In comparison, 70-80% of the LVS-vaccinated animals survived beyond 35 days post challenge.

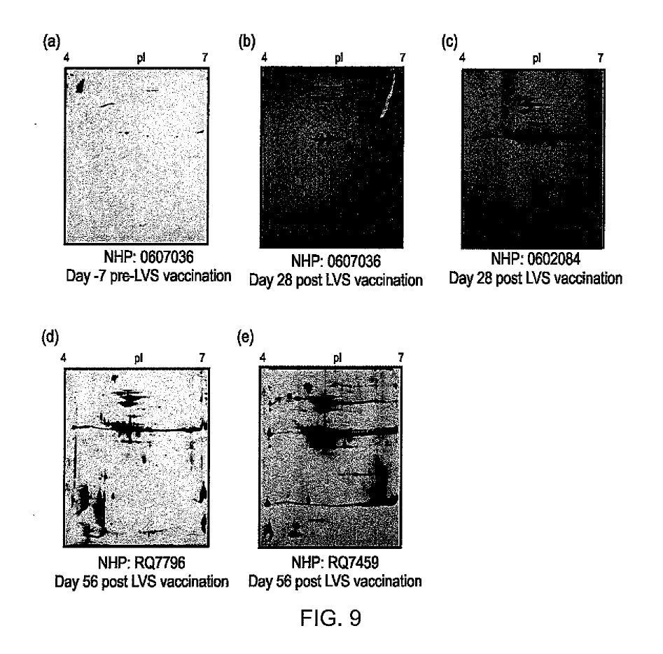

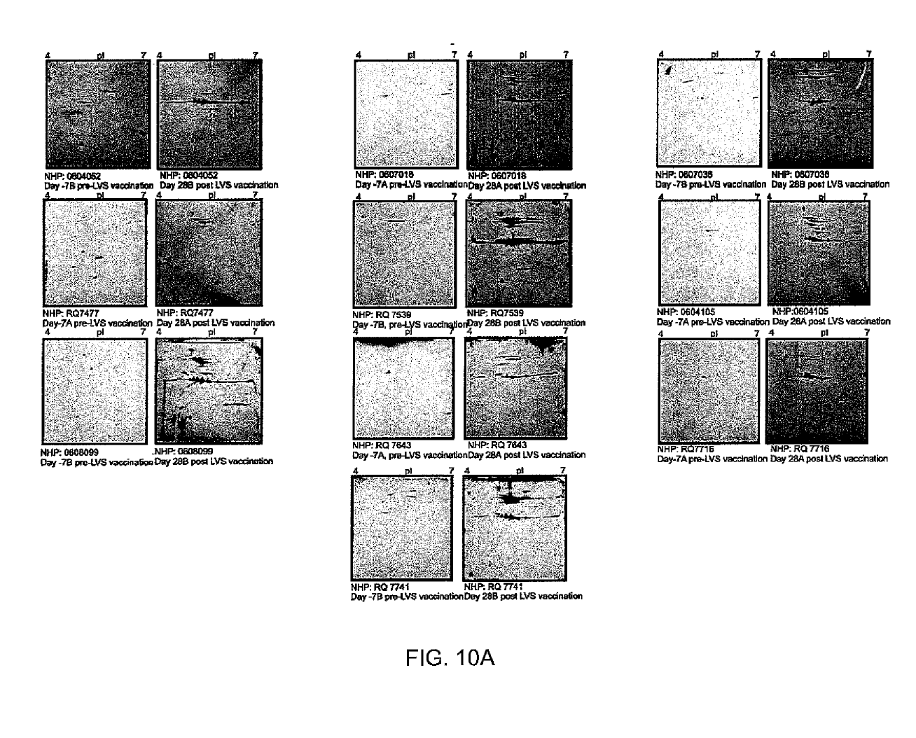

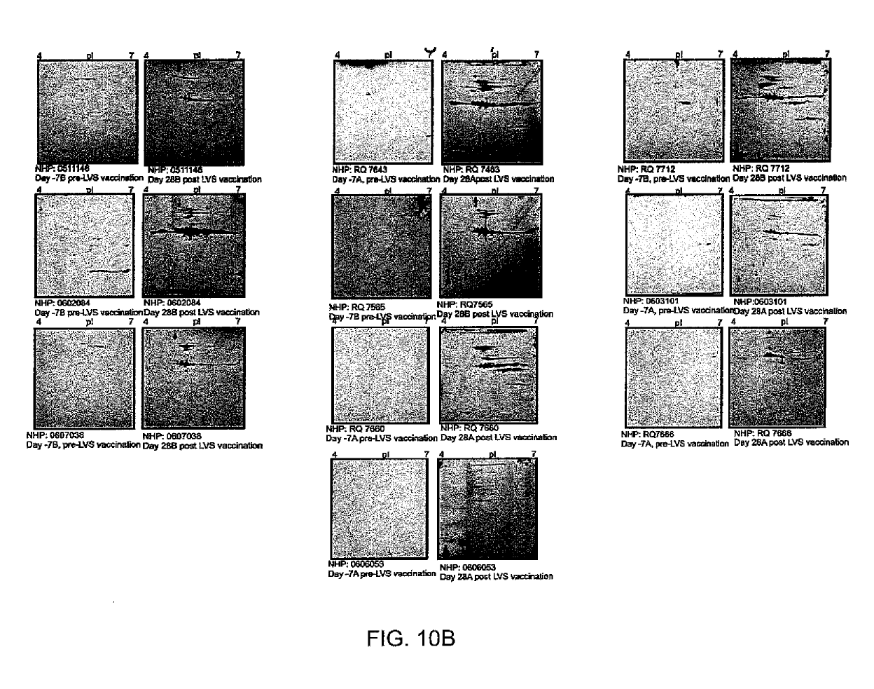

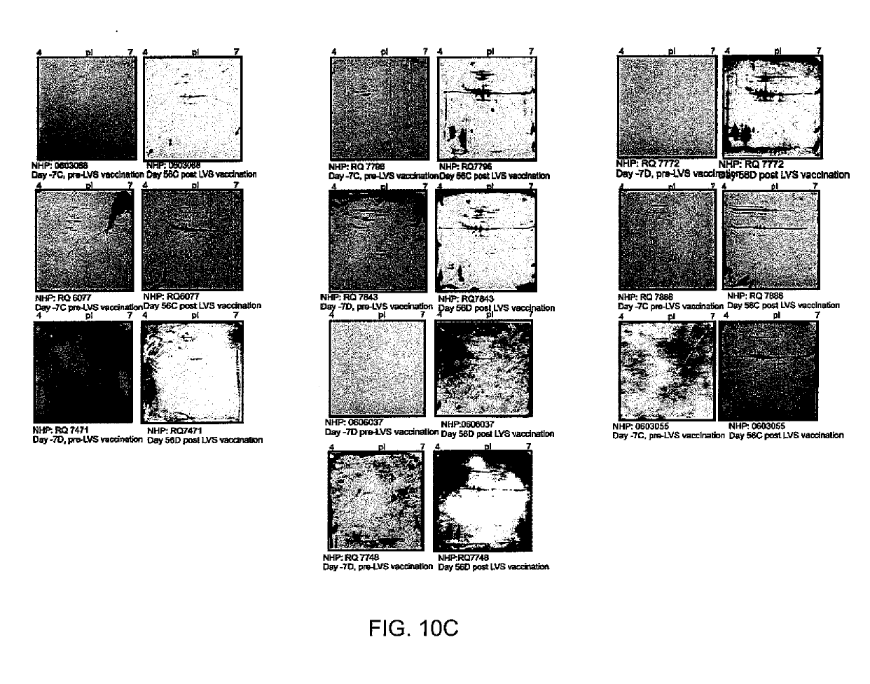

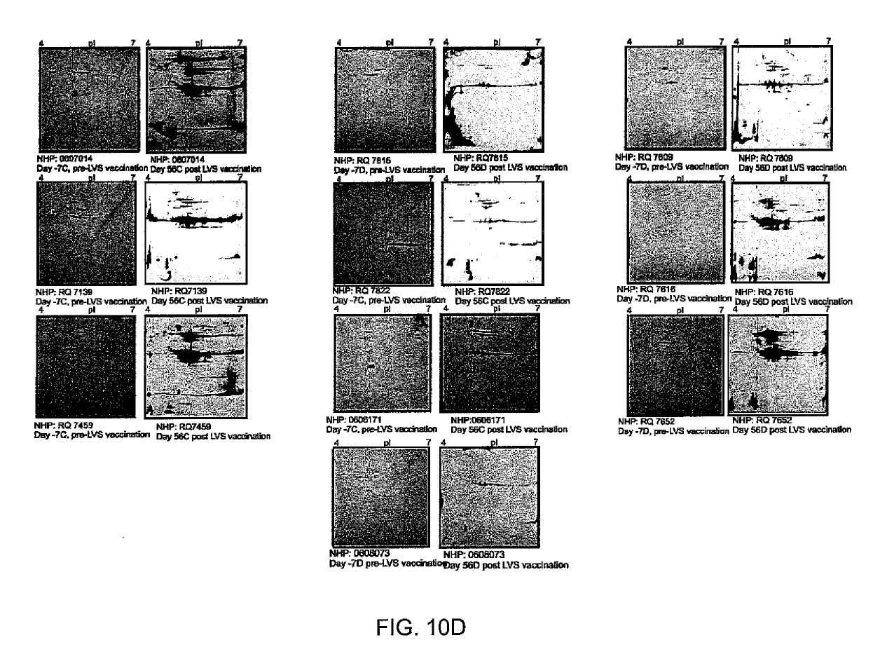

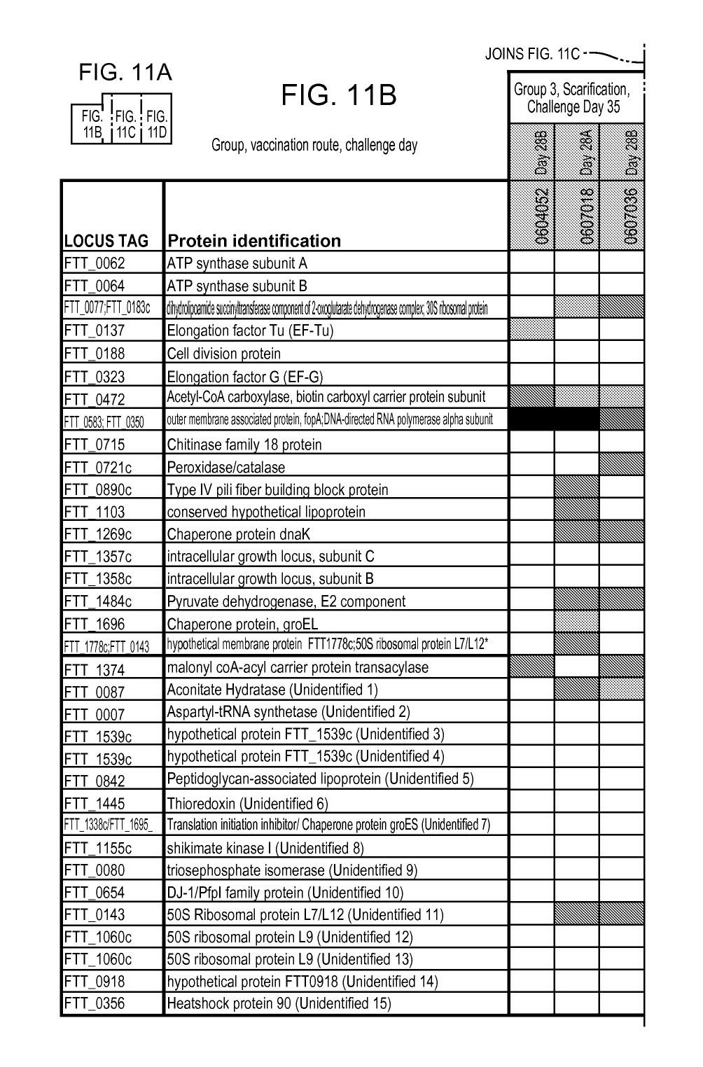

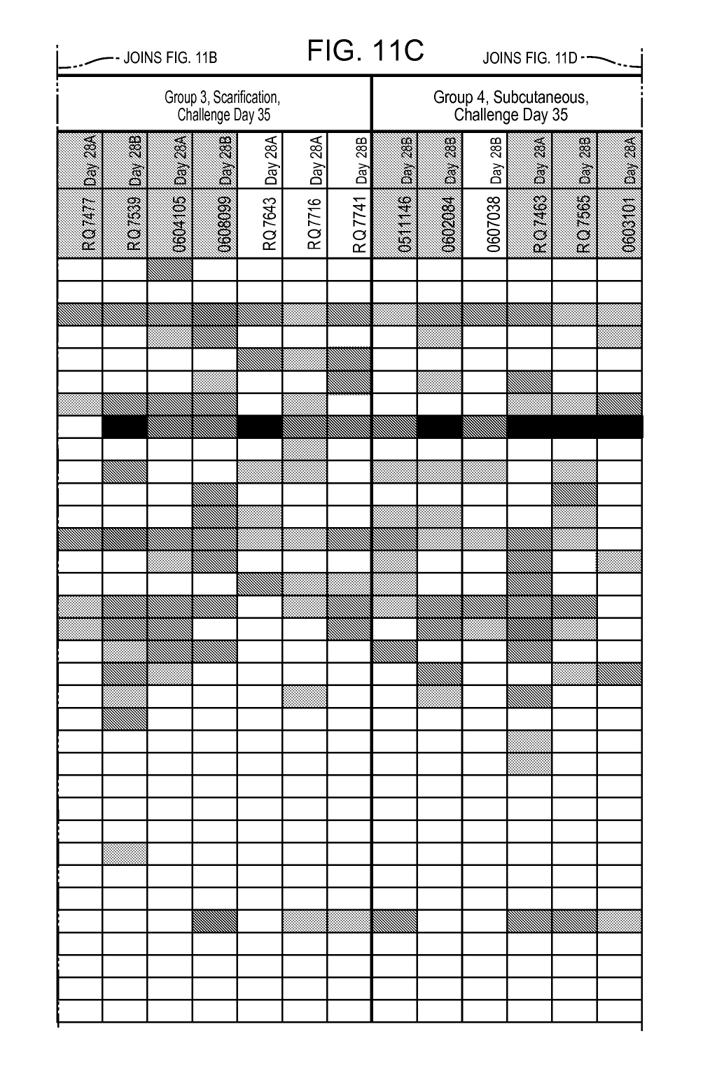

2D-PAGE and Western blotting was performed as described in Example 2. Representative 2D Western blots are shown in FIG. 9; full results are shown in FIG. 10. FIG. 9A is representative of Western blots probed with sera collected from animals pre-LVS vaccination. All sera collected from animals at either 28 or 56 days post LVS vaccination showed a marked increase in the number of regions of immunoreactivity and the intensity of immunoreactivity observed.

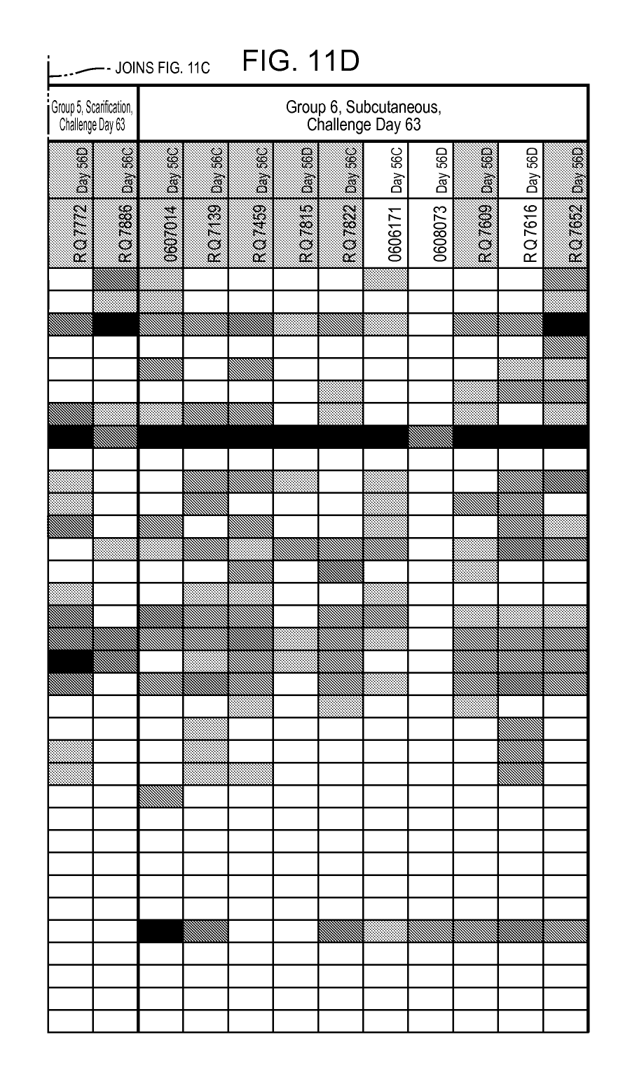

Immunoreactive areas observed on Western blots were aligned, where possible, with corresponding regions of protein staining on 2D-PAGE. The immunoreactive proteins were identified (see Example 3) and are shown in the matrix of immunoreactive proteins, in FIG. 11; due to the number of sera screened and space constraints, only data from post-vaccination sera are shown. Animals that survived subsequent SCHU S4 challenge are indicated in this figure by greyscale shading of the box indicating animal number, versus white for animals that succumbed to challenge. Blots probed with pre-vaccination sera showed minimal or no immunoreactivity (data not shown).

In general, the repertoire of immunoreactive proteins observed to be reactive with the post-vaccination NHP sera showed a great deal of overlap with the immunoreactive proteins identified in Example 3. Of particular note, outer membrane protein FopA (FTT_{—}0583) and DNA directed RNA polymerase A1 (FTT_{—}0350), which focus to the same protein spots on 2D-PAGE, were reactive with all post-vaccination sera. In addition, the proteins FTT_{—}0077 and FTT_{—}0183 were reactive with almost all of the sera screened. There appeared no clear pattern of immunoreactivity that distinguished animals vaccinated via different routes, nor survivors versus non-survivors of SCHU S4 challenge.

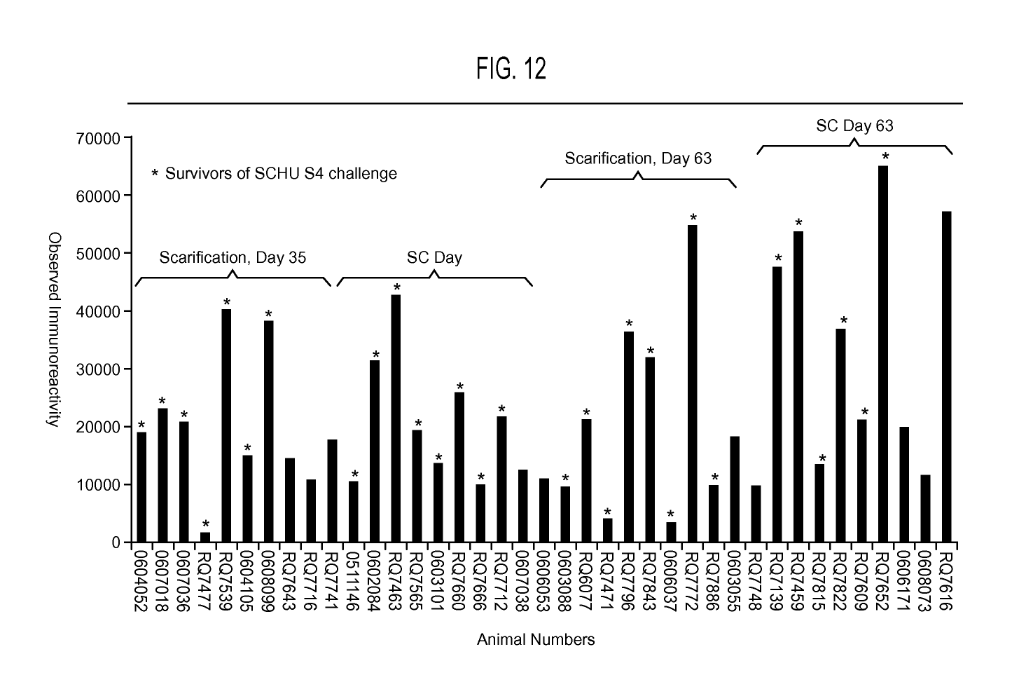

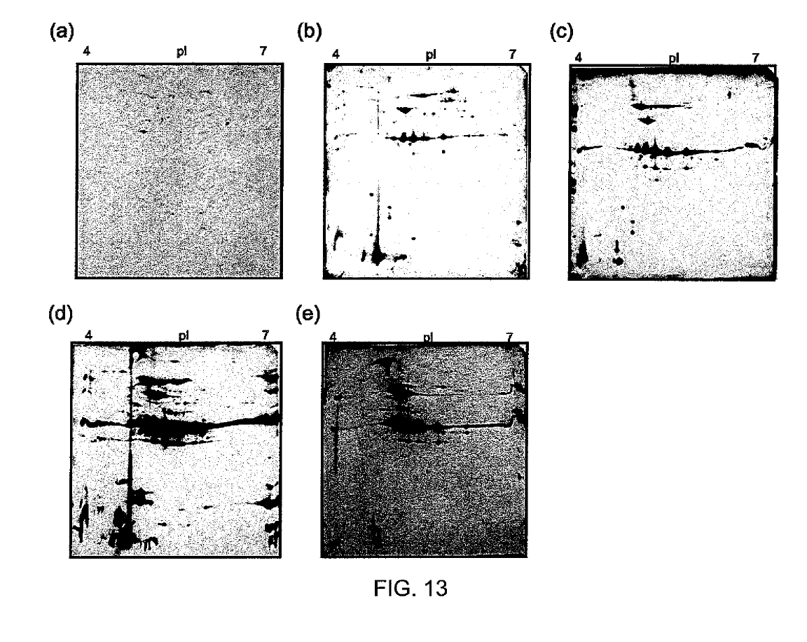

FIG. 12 shows a plot of total observed protein immunoreactivity from Western blots of sera from LVS-vaccinated animals. Within the chart, animals are grouped by route of vaccination and date of serum collection, in order to determine whether a relationship exists between total observed protein immunoreactivity and survivors of SCHU S4 challenge. The intensity of total immunoreactivity showed a large variation between individual animals, with no clear distinction between vaccinated animals that survived challenge and or those that succumbed to challenge with SCHU S4.

### Example 5

### Immunoproteomics in Rabbit Model of Tularemia

The rabbit model of tularemia used in this Example was developed by the Midwest Research Institute (MRI) in New Zealand white rabbits.

Serum samples were provided by the Midwest Research Institute (MRI Project No. 110645.1.006: Evaluation of DVC F. tularensis Live Vaccine Strain (LVS) Efficacy in the New Zealand White Rabbit against Aerosol Challenge with DVC F. tularensis SCHU S4). A comprehensive characterization of LVS vaccination in New Zealand White Rabbits was established, with two routes of vaccination (Scarification and SC) and challenge dates either 42 or 63 days post-LVS vaccination. The study design is summarized in Table 5.

In total, 84 sera were provided, comprising of 64 sera from LVS-vaccinated rabbits (paired pre- and post-vaccination sera), eight sera from sham-vaccinated animals and sera from two control animals. The post-LVS vaccination sera were drawn either 42 days or 63 days post vaccination.

TABLE 5
Evaluation of DVC F. tularensis Live Vaccine Strain (LVS) Efficacy
in the New Zealand White Rabbit against Aerosol Challenge with DVC
F. tularensis SCHU S4 Study design.

| | | | | | | Challenge |
|---|---|---|---|---|---|---|
| | | | | | Chal- | dose |
| | | | Dose | | lenge | (SCHU |
| Group | # Rabbits | Vaccine | (cfu) | Route | day | S4) |
| 1 | 8 | LVS | 8 × 10⁷ | Scarification | 42 | 10³ |
| 2 | 8 | LVS | 8 × 10⁷ | SC | 42 | 10³ |
| 3 | 4 | Saline | NA | Scar/SC | 42 | 10³ |
| 4 | 8 | LVS | 8 × 10⁷ | Scarification | 63 | 10³ |
| 5 | 8 | LVS | 8 × 10⁷ | SC | 63 | 10³ |
| 6 | 4 | Saline | NA | Scar/SC | 63 | 10³ |
| 7 | 2 | None | NA | NA | NA | NA |

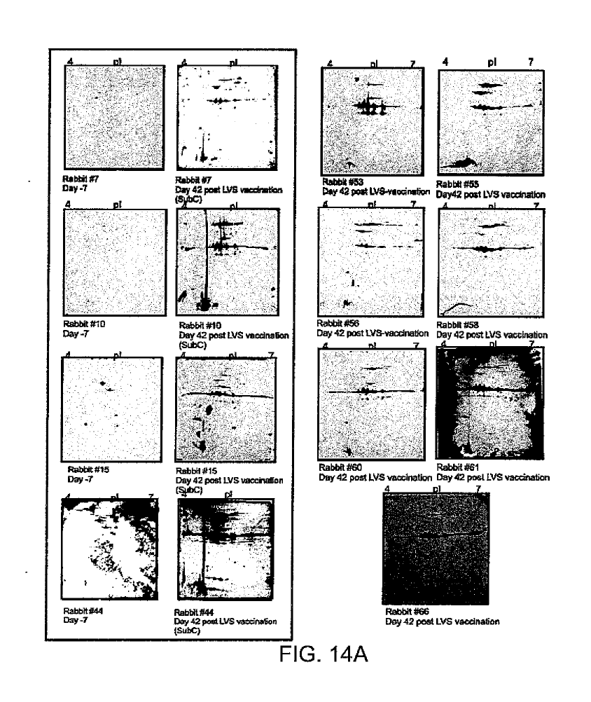

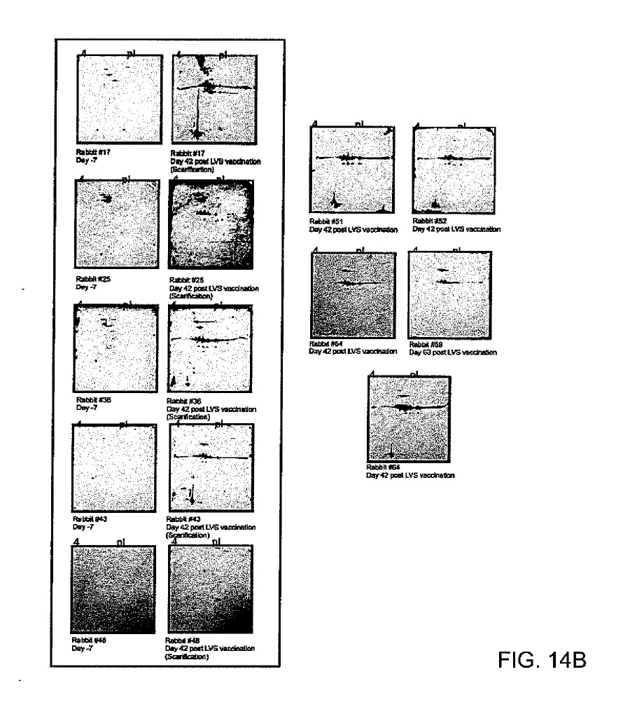

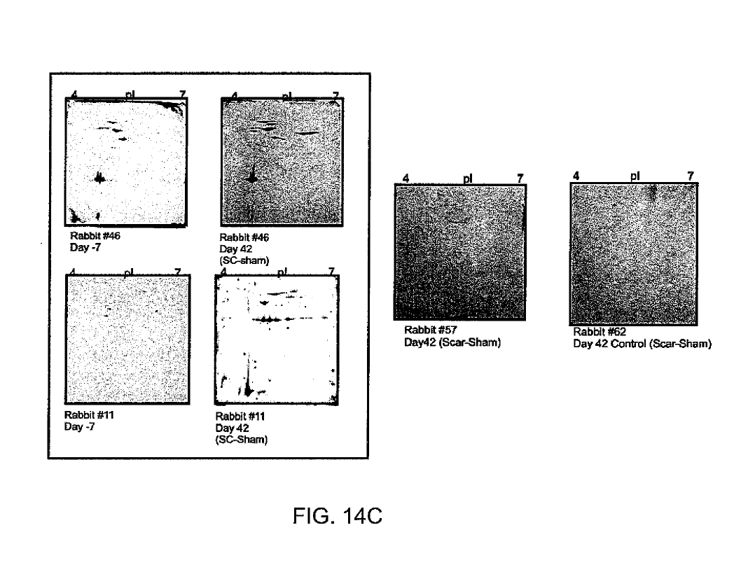

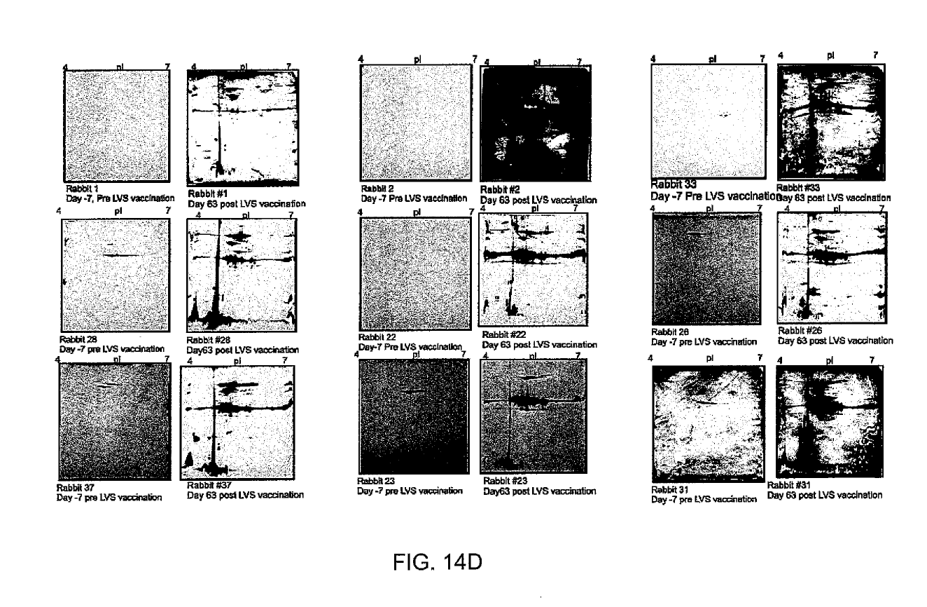

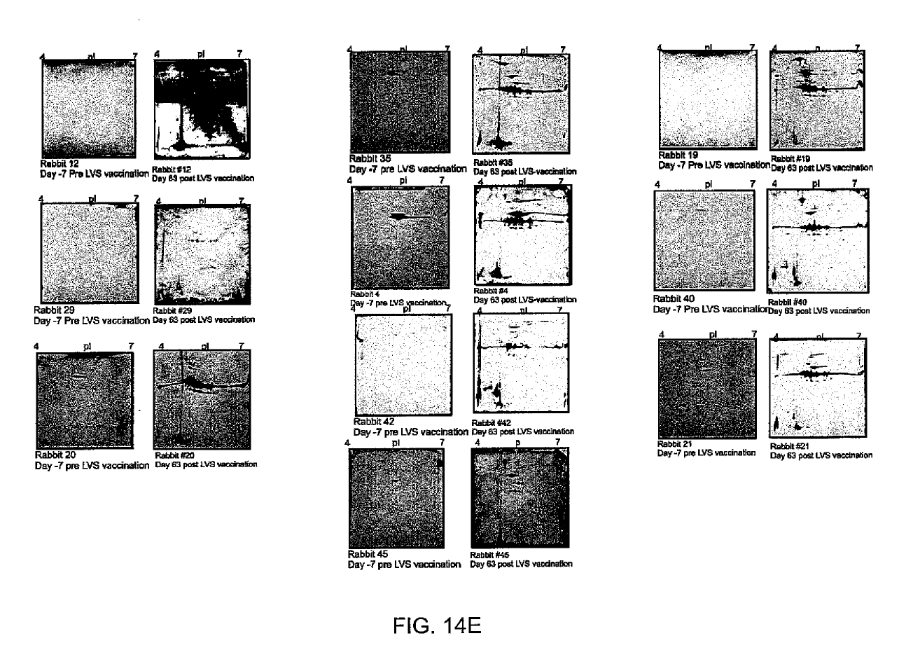

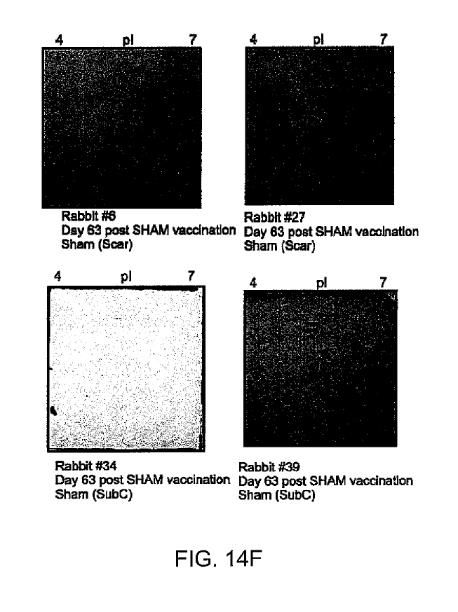

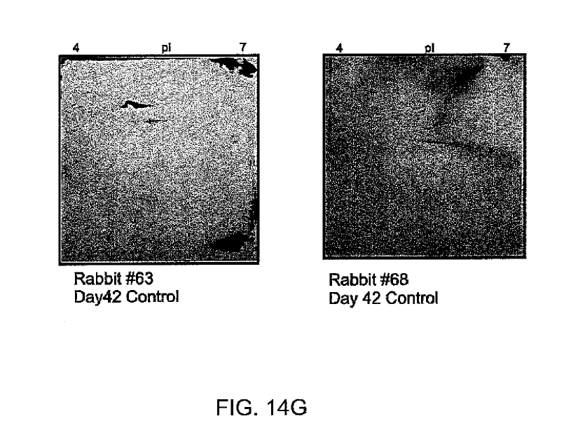

Pre- and post-vaccination sera from each animal in the study groups listed in Table 5 were screened by 2D Western blotting as described in Example 2. FIG. 13 shows representative blots from this series, showing a blot probed with serum drawn pre-LVS vaccination (FIG. 13A), and 42 or 63 post LVS vaccination by either SC or scarification (FIG. 13B-E). Full results of Western blots are shown in FIG. 14. The Western blot probed with sera drawn pre-vaccination showed only weak immunoreactivity towards a small number of proteins. This was typical for all pre-vaccination sera screened. In addition, blots probed with sera from sham-vaccinated or control animals showed few, if any, weakly reactive areas of immunoreactivity. In contrast, all Western blots probed with sera drawn from animals post-LVS vaccination showed a marked increased in the observed total observed immunoreactivity and the number of immunoreactive proteins recognized.

Western blot images were aligned with equivalent protein stained gels, and regions that aligned with areas of protein staining were excised and the corresponding proteins identified by nLC-MS/MS of their tryptic digests (see Example 3). From this, a total of 25 immunoreactive proteins were identified. As in previous Examples, some intense areas of immunoreactivity corresponded to more than one protein spot (e.g., FTT_{—}0077/FTT_{—}0183), several immunoreactive protein spots focussed to multiple areas on 2DE (e.g., FTT_{—}0583) and in some instances immunoreactivity did not correspond with detectable protein staining. In the latter case, it is important to note that immunoreactivity is not necessarily proportional to protein concentration, and the corresponding immunoreactive protein may be beyond the limits of silver staining detection in the 2D-PAGE approach.

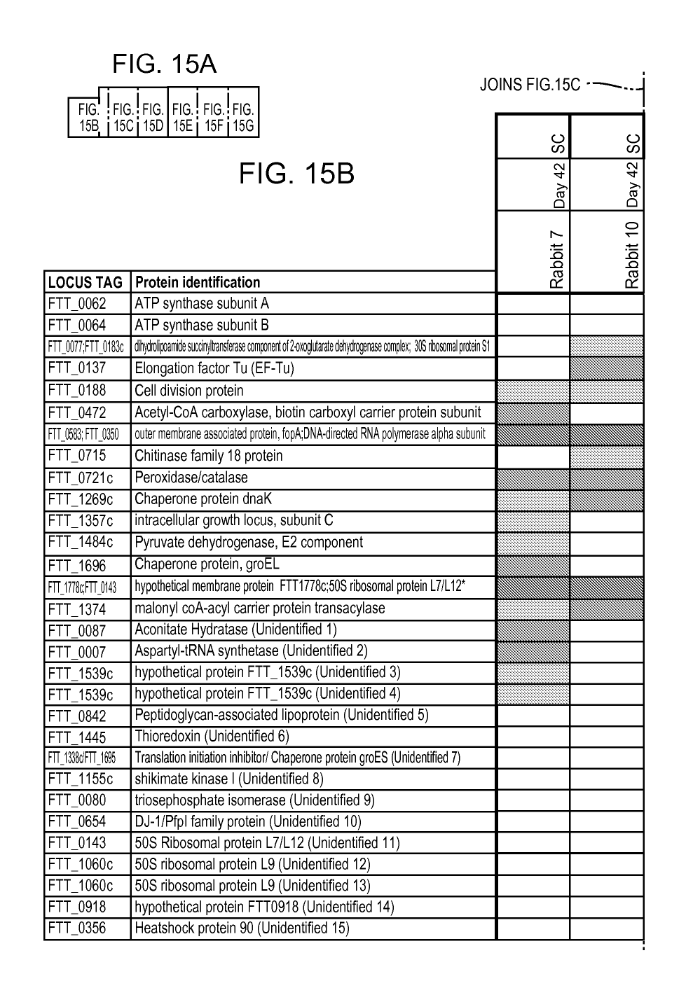

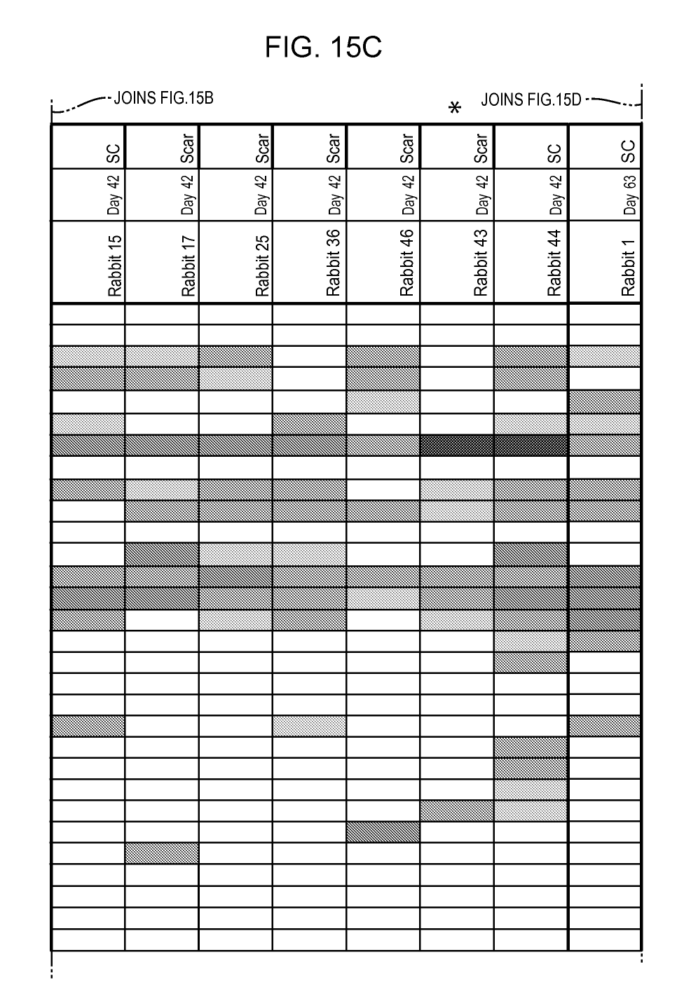

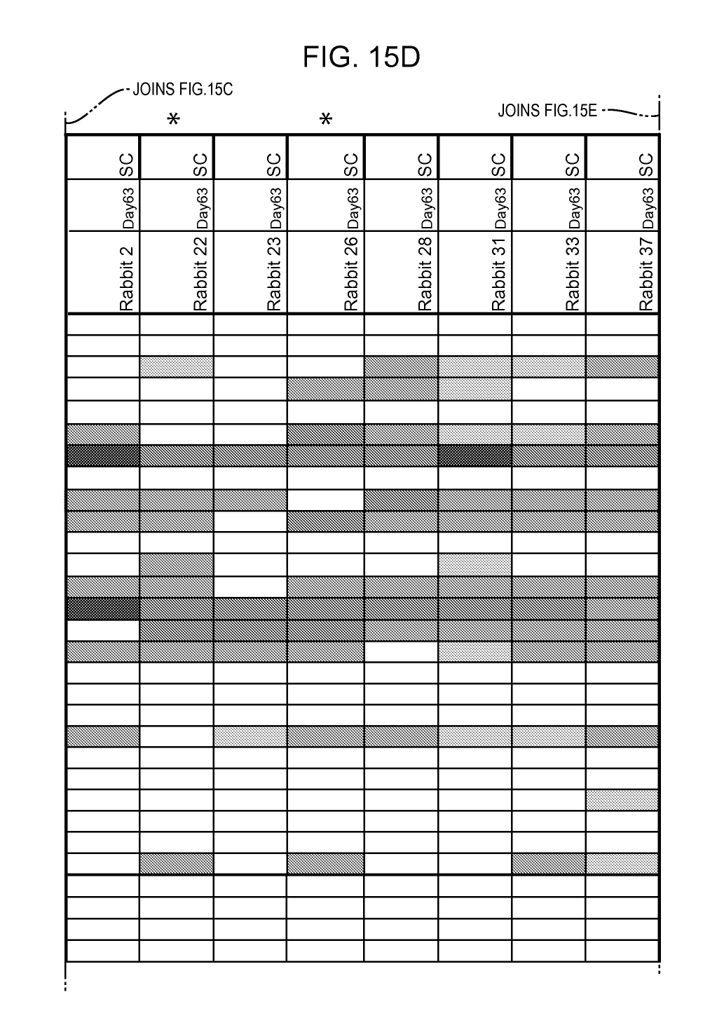

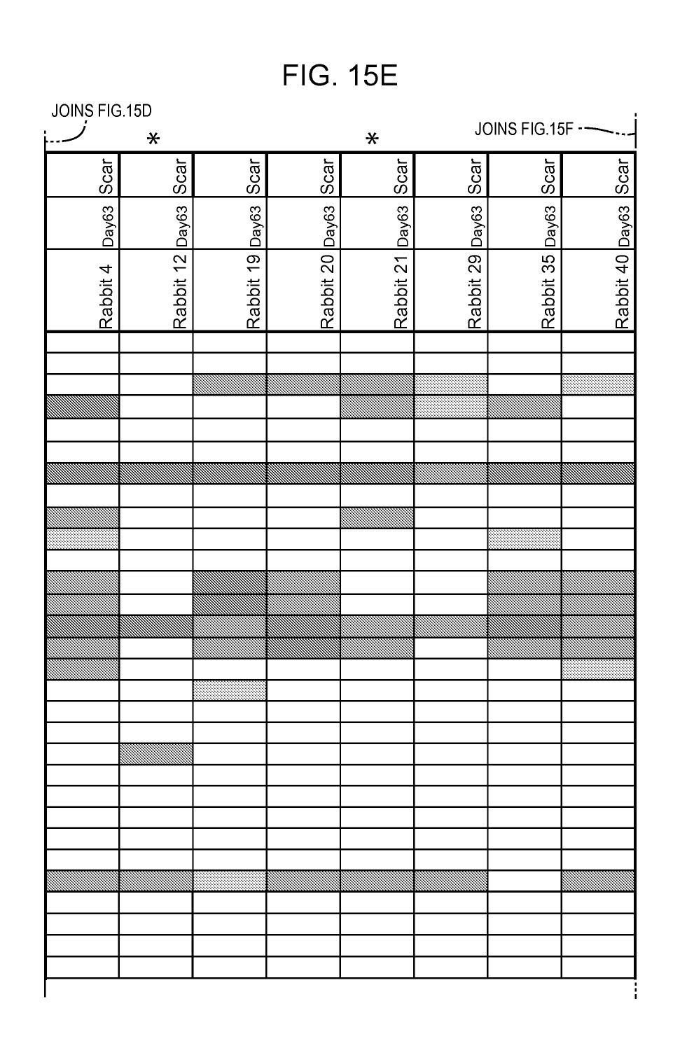

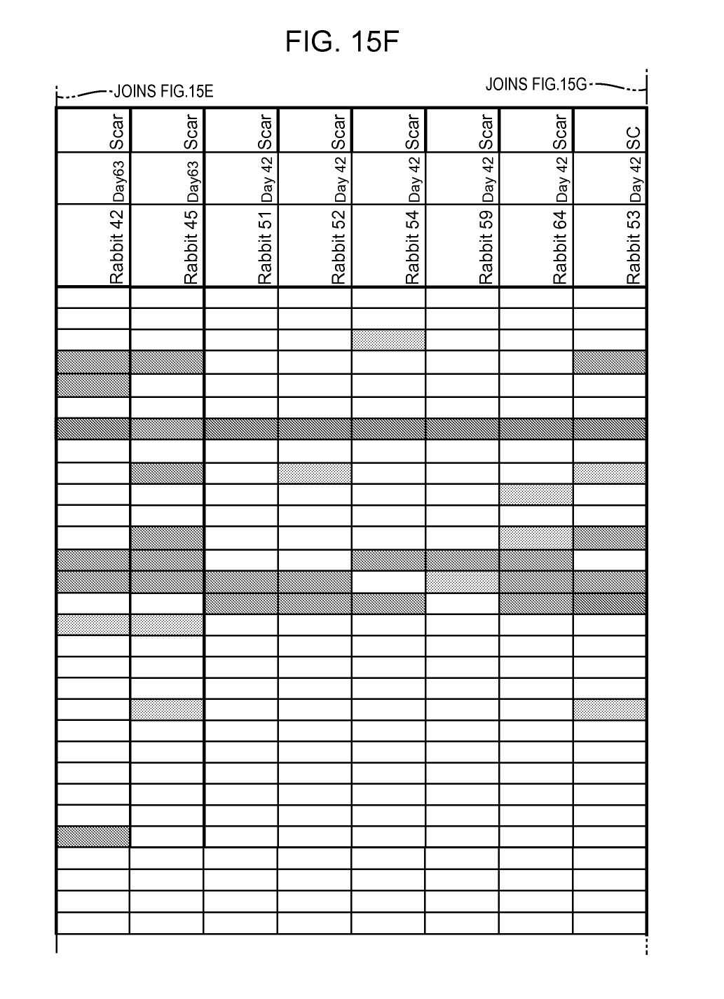

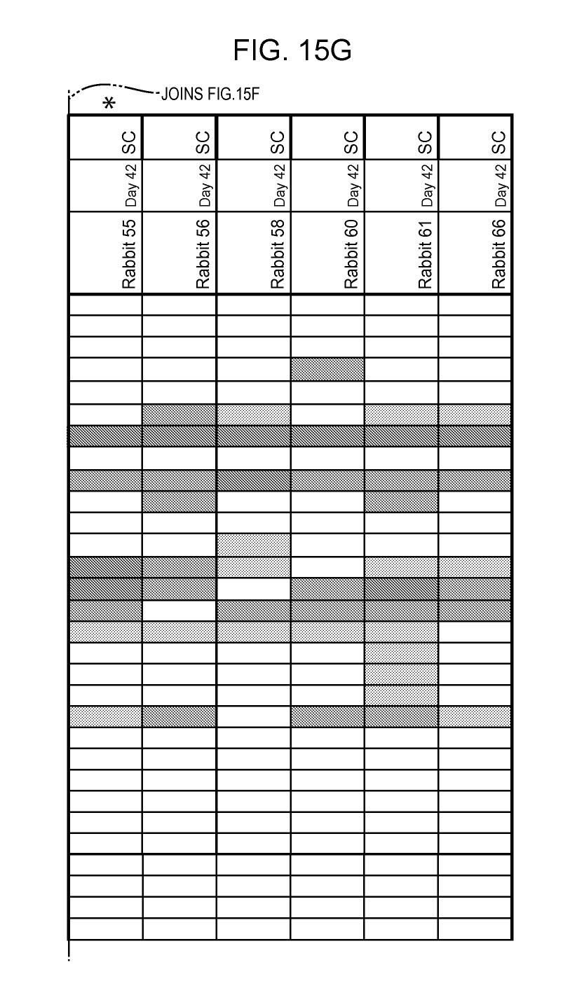

The identified immunoreactive proteins are shown as a matrix of immunoreactive proteins in FIG. 15, and the details of mass spectrometry identification are summarized in Example 2. Due to the large number of sera screened, data from pre-vaccination sera, sham-vaccinated animals and control animals have been omitted. As indicated by an asterisk in FIG. 15, only six animals survived aerosol challenge with SCHU S4 (MRI Project No. 110645.1.006: Evaluation of DVC F. tularensis Live Vaccine Strain (LVS) Efficacy in the New Zealand White Rabbit against Aerosol Challenge with DVC F. tularensis SCHU S4). Of the six animals, one from each of the SC vaccination groups survived SCHU S4 challenge. A further three animals vaccinated by scarification survived challenge at 42 days post-vaccination, and a single animal also vaccinated by scarification, survived challenge at 63 days post vaccination.

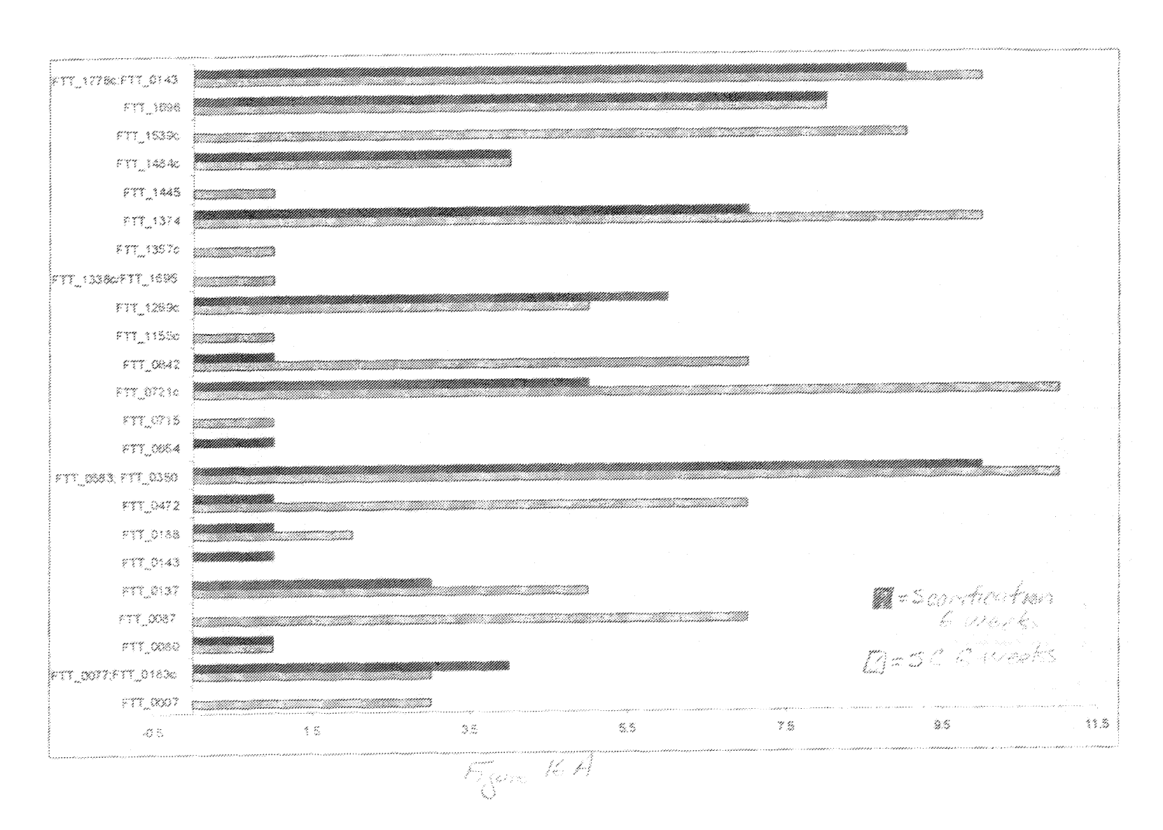

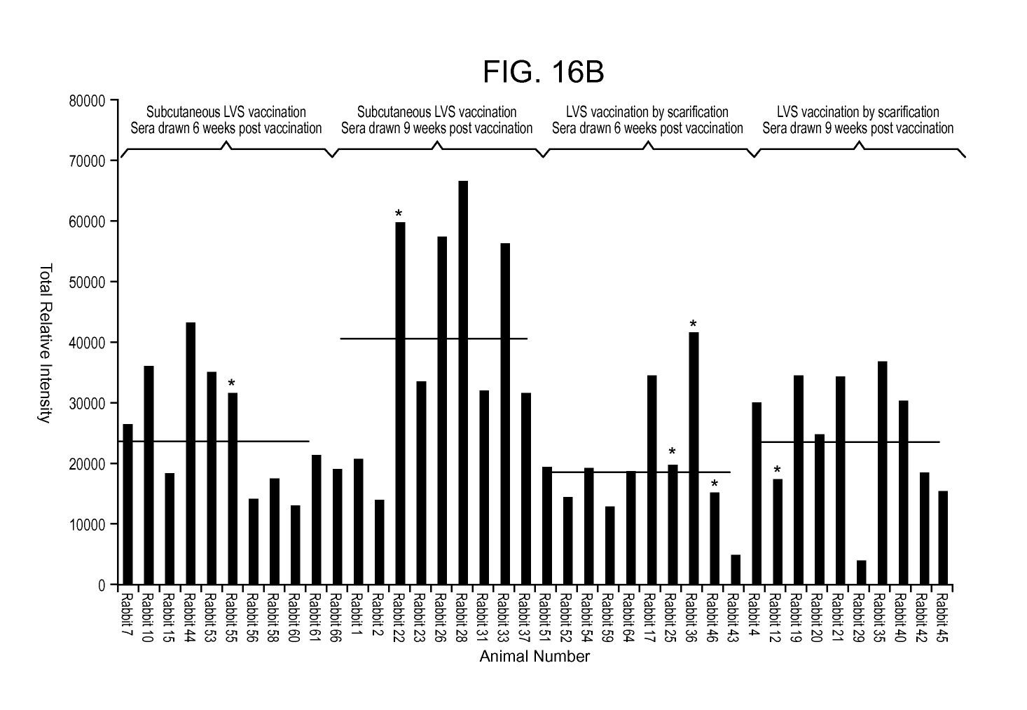

The number of immunoreactive proteins identified for each LVS-vaccinated animal varied with, on average, ten immunoreactive proteins identified from blots probed with sera from both SC LVS-vaccinated rabbits drawn at both 42 and 63 days post-vaccination. By comparison, the average number of identified immunoreactive proteins from blot probed with sera from rabbits vaccinated by scarification was six (42 days post-vaccination) and seven (63 days post-vaccination). From the matrix of immunoreactive proteins, it can be seen that some proteins are immunoreactive with many of the post-vaccination sera screened. In order to gauge how frequently certain proteins were recognized by immune sera, the frequency with which each immunoreactive protein was observed was plotted as a bar chart (FIG. 16). Of note, the proteins FTT_{—}1778c/FTT_{—}0143, FTT_{—}1696, FTT_{—}1539c, FTT_{—}1374, FTT_{—}0721c, and FTT_{—}0583/FTT_{—}0350 were reactive with more than half of the post-LVS vaccination sera screened in this study. In particular, seven proteins, FTT_{—}0715, FTT_{—}1357c, FTT_{—}1539c, FTT_{—}1445, FTT_{—}1338c, FTT_{—}1695 and FTT_{—}1155c were only observed to be reactive with sera drawn from subcutaneously vaccinated rabbits. Other proteins such as Catalase/peroxidase (FTT_{—}0721c) were observed to be reactive with all sera from rabbits vaccinated SC and with only half of the sera drawn from rabbits vaccinated by scarification. Interestingly, Outer membrane protein FopA (FTT_{—}0583) and DNA directed RNA polymerase (FTT_{—}0183) were observed to be reactive with all post-LVS vaccination sera screened, regardless of the route of LVS vaccination or day post-vaccination that sera was drawn.

In addition to variation in the repertoire of immunoreactive proteins, sera from LVS-vaccinated rabbits showed variation in the total observed intensity of protein immunoreactivity. The total intensity of immunoreactivity was summed for each serum screened and subsequently plotted in bar chart format, shown in FIG. 16. The bar chart is grouped by vaccination route, and date sera were drawn post-LVS vaccination, with a horizontal bar indicating the mean intensity value for a particular group. From this graph, it is apparent that the total intensity of protein immunoreactivity varies between animals and the mean values vary between the vaccination groups. The mean intensity of protein immunoreactivity values are higher for the SC LVS-vaccinated animals, compared with the mean for groups of animals LVS-vaccinated by scarification. Within the SC-vaccinated groups of animals, a small, but not statistically significant increase in immunoreactivity in for SC vaccinated animals between 42 and 63 days post vaccination was observed. By comparison, the mean observed intensity of immunoreactivity for rabbits LVS-vaccinated by scarification did not differ greatly between sera drawn at 42 or 63 days post-vaccination. Animals that survived challenge with SCHU S4 are indicated by asterisk (*) in FIGS. 15 and 16. No clear pattern was evident from the simple analysis of these data; surviving animals did not appear to generate antibodies towards specific proteins, nor a greater intensity of reactivity than those that succumbed to challenge (FIGS. 15 and 16).

From the human immunoproteomics work described in Example 3, eleven proteins were downselected as commonly reactive antigens, reactive with both human patient and vaccinee sera, with a minimum frequency of 30%. These include dihydrolipoamide succinyltransferase component of 2-oxoglutarate dehydrogenase complex (FTT_{—}0077), 50S ribosomal protein L7/L12 (FTT_{—}0143), 30S ribosomal protein S1 (FTT_{—}0183), DNA-directed RNA polymerase alpha subunit (FTT_{—}0350), Acetyl-CoA carboxylase (FTT_{—}0472), Outer membrane associated protein FopA (FTT_{—}0583), Peroxidase/catalase FTT_{—}0721c), Chaperone protein DnaK (FTT_{—}1269c), Pyruvate dehydrogenase E2 component (FTT_{—}1484c), Chaperone protein groEL (FTT1696), Hypothetical membrane protein (FTT_{—}1778c). When comparing the repertoire of immunoreactive proteins identified from 2D Western blots probed with sera from LVS-vaccinated rabbits and non-human primates, with those identified from human patients and LVS vaccinees, the same eleven proteins were observed to be reactive with sera from all species. Table 6 lists these proteins and the frequency with which they were observed to be reactive with sera from each animal species. For the NHP immunoproteomics, this was subdivided into the frequency of reactivity with those animals surviving challenge and those that succumbed to challenge with SCHU S4.

TABLE 6
Francisella proteins found to be reactive with sera from LVS-vaccinated humans and
animal models, and infected humans. The locus tag refers to the SCHU S4 genome sequence;
for each group the number of subjects and the number of sera in which reactivity towards a
specific protein was observed. For the NHP study, the frequency of reactivity with animals
surviving challenge, and those succumbing to SCHU S4 challenge is indicated.

| | | Vaccine | Vaccine | | | | |
|---|---|---|---|---|---|---|---|
| | | NDBR lot | DVC lot | Type B | Type A | | |
| | | 11 LVS | 17 LVS | convalescent | convalescent | NHP | Rabbit |
| Locus tag | Protein Name | (n = 8) | (n = 8) | (n = 12) | (n = 12) | (n = 40)* | (n = 40) |
| FTT_0077 | dihydrolipoamide | 8 | 7 | 11 | 8 | 26, 7 | 27 |
| | succinyltransferase | | | | | | |
| | component of 2- | | | | | | |
| | oxoglutarate | | | | | | |
| | dehydrogenase complex | | | | | | |
| FTT_0143 | 50S ribosomal protein | 8 | 6 | 8 | 10 | 19, 2 | 38 |
| | L7/L12 | | | | | | |
| FTT_0183 | 30S ribosomal protein S1 | 7 | 5 | 7 | — | 26, 7 | 27 |
| FTT_0350 | DNA-directed RNA | 5 | 3 | 4 | — | 29, 10 | 40 |
| | polymerase alpha subunit | | | | | | |
| FTT_0472 | Acetyl-CoA carboxylase, | 6 | 3 | 8 | 7 | 23, 1 | 15 |
| | biotin carboxyl carrier | | | | | | |
| | protein subunit | | | | | | |
| FTT_0721c | Peroxidase/catalase | 5 | 5 | 6 | 7 | 13, 7 | 27 |
| FTT_1269c | Chaperone protein dnaK | 5 | 7 | 5 | 8 | 24, 7 | 21 |
| FTT_1484c | Pyruvate dehydrogenase | 4 | 3 | 3 | 10 | 25, 6 | 16 |
| | E2 component | | | | | | |
| FTT_1696c | Chaperone protein, | 4 | 6 | 12 | 9 | 20, 4 | 31 |
| | groEL | | | | | | |
| FTT_1778c | Hypothetical membrane | 8 | 6 | 8 | 5 | 19, 2 | 38 |
| | protein FTT_1778c | | | | | | |
| FTT_0583 | Outer membrane | 5 | 3 | 4 | — | 29, 10 | |
| | associated protein | | | | | | |
| | fopA | | | | | | |

*30 survivors, 10 non-survivors of SCHU S4 challenge

### Example 6

### Immunoproteomics in Murine Model of Tularemia

The murine model of tularemia used in this Example has been described in the literature, and is the most widely used tularemia model. However, concerns regarding how applicable findings in mice are to humans exist, not least because LVS remains virulent for mice. For example, F. tularensis LVS ATCC 29684 inoculated intradermally (ID) elicits a similar sub-lethal infection in the skin, liver, and spleen of both BALB/c and C57BL/6 mice that persists for approximately 2 weeks. LVS also differs in the ability to protect mice from challenge with type A strains of the pathogen, depending upon the genetic background of the mouse. LVS infection renders BALB/c mice immune to a subsequent systemic challenge with >100 LD₅₀ of a virulent type A strain of F. tularensis, it fails to protect C57BL/6 mice from a 100-fold smaller challenge.

Earlier work compared the immunoproteomes of the two LVS-vaccinated mouse strains aimed to determine whether successful immunization of BALB/c mice with lot 16 LVS results in seroconversion to a unique subset of immunoreactive proteins (Twine et al., 2010). Twenty-eight proteins were observed to be immunoreactive with sera from successfully and unsuccessfully vaccinated BALB/c mice, including the following proteins observed to be reactive with sera from other animal modes of tularaemia: Outer membrane associated protein FopA (FTT0583), Chaperonin GroEL (FTT1696), Dihydrolipoamide succunyl transferase component of 2-oxoglutarate dehydrogenase complex (FTT0077), and acetylCoA carboxylase (FTT_{—}0472).

Intranasal and Subcutaneous Vaccination of BALB/c Mice with LVS

Development of the murine model of tularemia at the National Research Council Canada included a direct comparison of routes of LVS vaccination (Lot 17 LVS), including subcutaneous (SC) and intranasal (IN) of BALB/c mice. For each vaccination route, a cage of mice (n=5) were vaccinated (IN dose was 1×10³ cfu; SC dose was 1×10⁴ cfu) and sera drawn four weeks post-vaccination. Immunoreactive proteins were detected and identified as described in Examples 2 and 3.

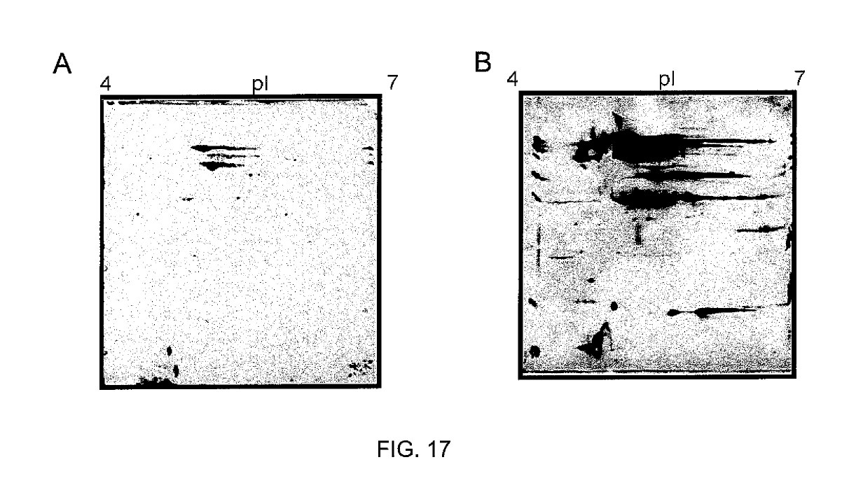

The results (FIG. 17) show that selected marker proteins of the present invention are reactive with post-vaccination sera, regardless of the route of vaccination. Specifically, the immunoreactive proteins identified are listed below, based on the route of vaccination.

Intranasal vaccination: the proteins dihydrolipoamide succinyltransferase (FTT_{—}0077), 30S ribsosomal protein S1 (FTT_{—}0183), GroEL (FTT_{—}1696), DnaK (FTT_{—}1296c), Acetyl CoA carboxylase (FTT_{—}0472) were reactive with sera from all vaccinated mice screened. The protein Catalase (FTT_{—}721c) was reactive with all but one of the sera screened. In addition, the outer membrane protein FopA (FTT_{—}0583) was reactive with some of the sera screened.

Subcutaneous vaccination: the proteins GroEL (FTT_{—}1696) and DnaK (FTT_{—}1269c) were observed to be reactive with all sera screened. In addition, the proteins dihydrolipoamide succinyltransferase (FTT_{—}0077) and 30S ribsosomal protein S1 (FTT_{—}0183) were reactive with the majority of sera drawn.

Comparison of the Profile of Immunoreactive Proteins with Antisera Generated by Subcutaneous Vaccination of BALB/c Mice with LVS and an LVS ΔIgIC Mutant.

To compare markers from a protected and an unprotected mouse, BALB/c mice that can be successfully vaccinated with LVS were also vaccinated with a mutant of LVS (LVS ΔIgIC) that is further attenuated and does not protect BALB/c mice from intradermal challenge with virulent SCHU S4. Comparison of the repertoire of immunoreactive proteins from successfully and unsuccessfully vaccinated mice has the potential to reveal antigenic proteins that are markers of protective vaccination. Vaccination was performed as described above (SC vaccination, dosage for LVS and mutant strains was 1×10⁴ cfu), and detection and identification of immunoreactive proteins was performed as described in Examples 2 and 3.

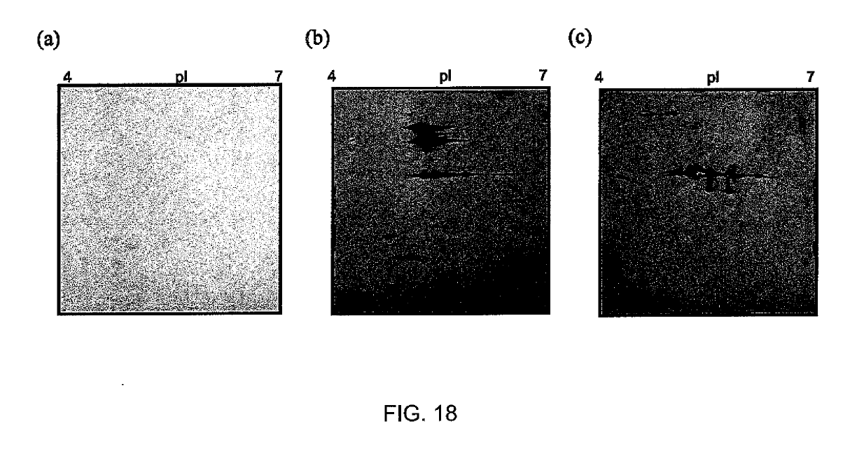

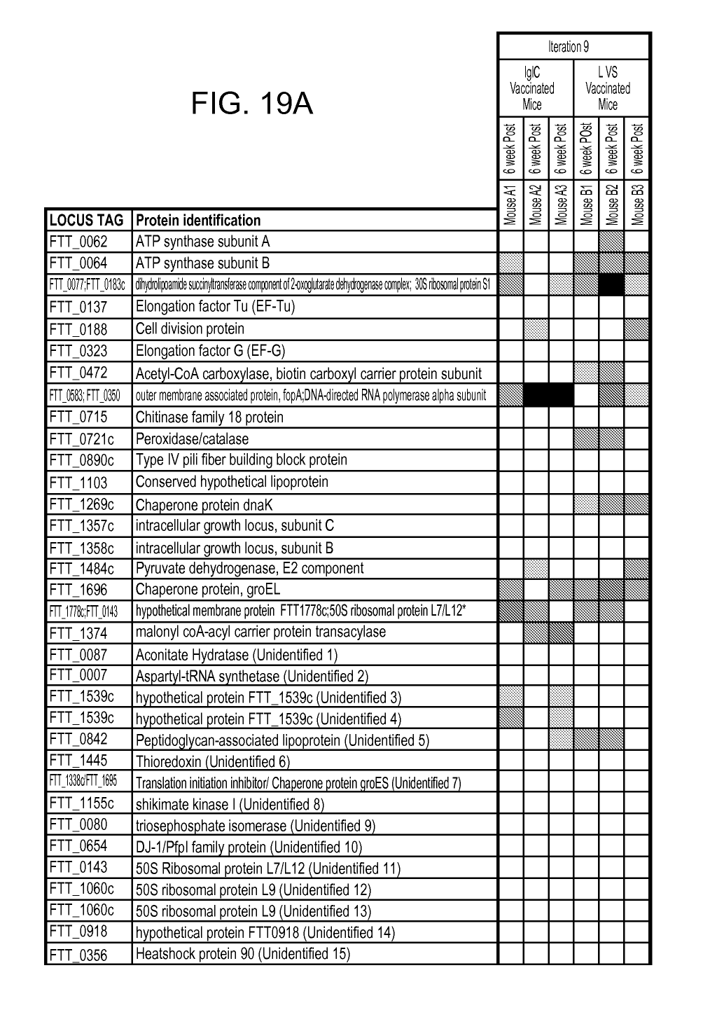

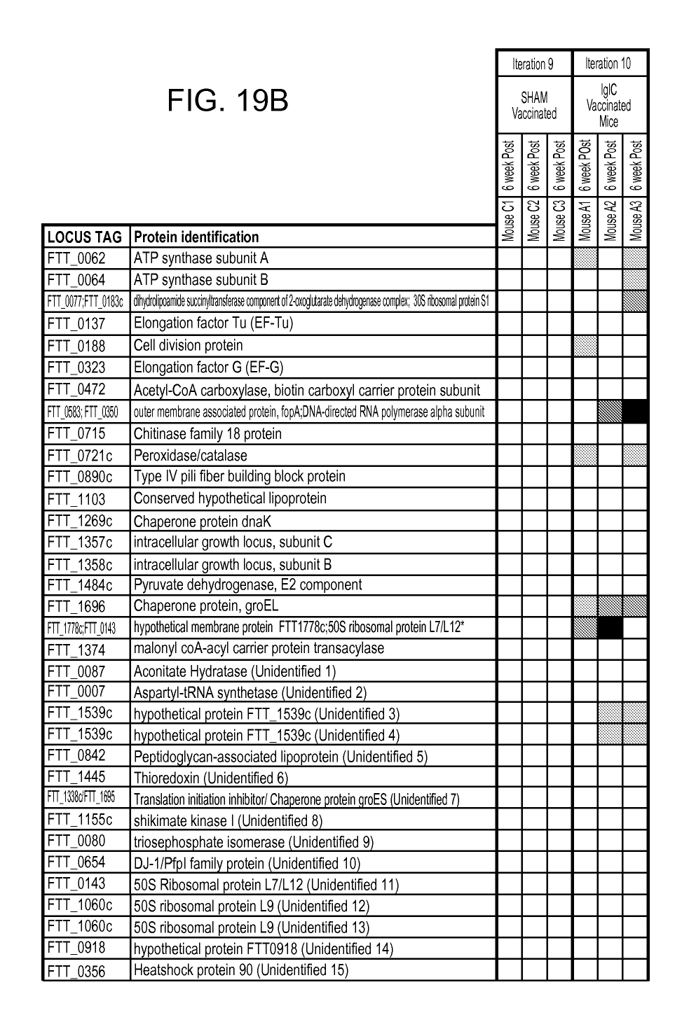

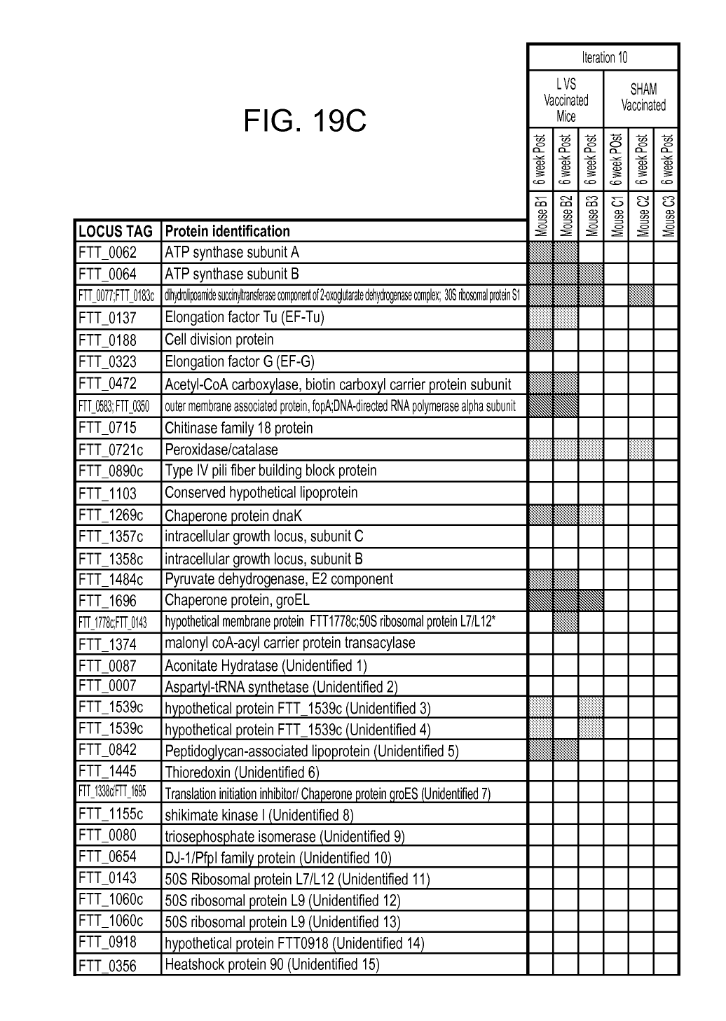

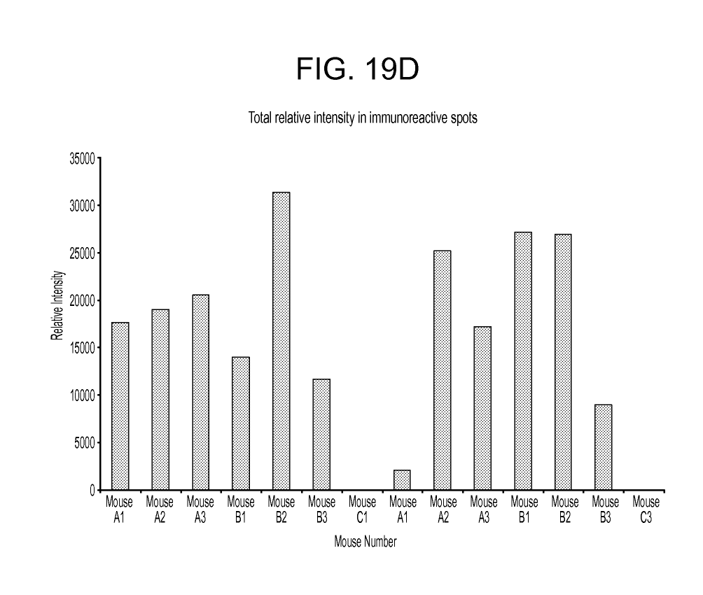

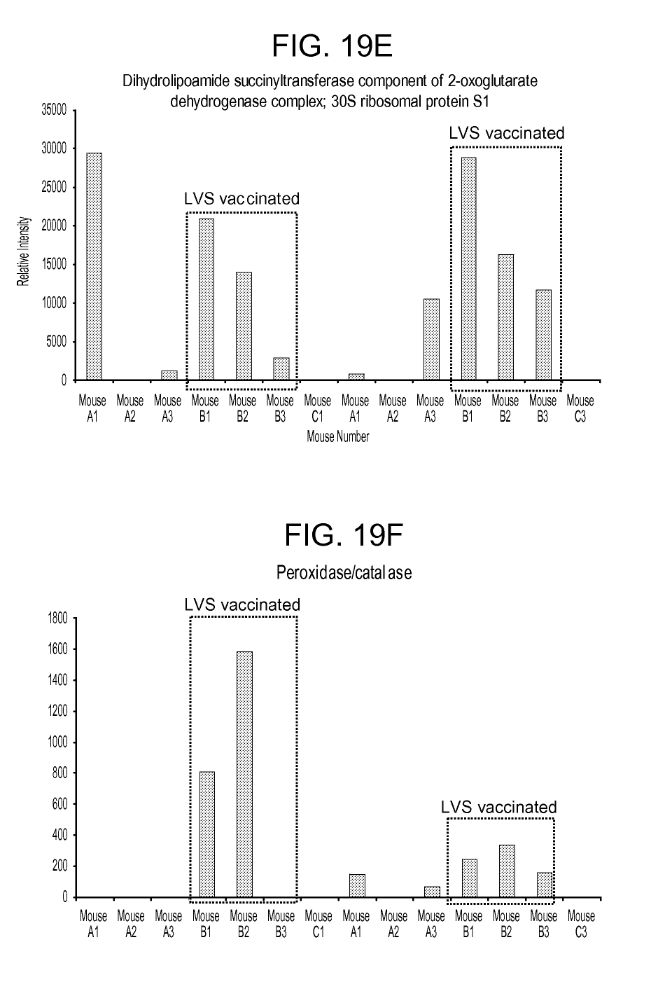

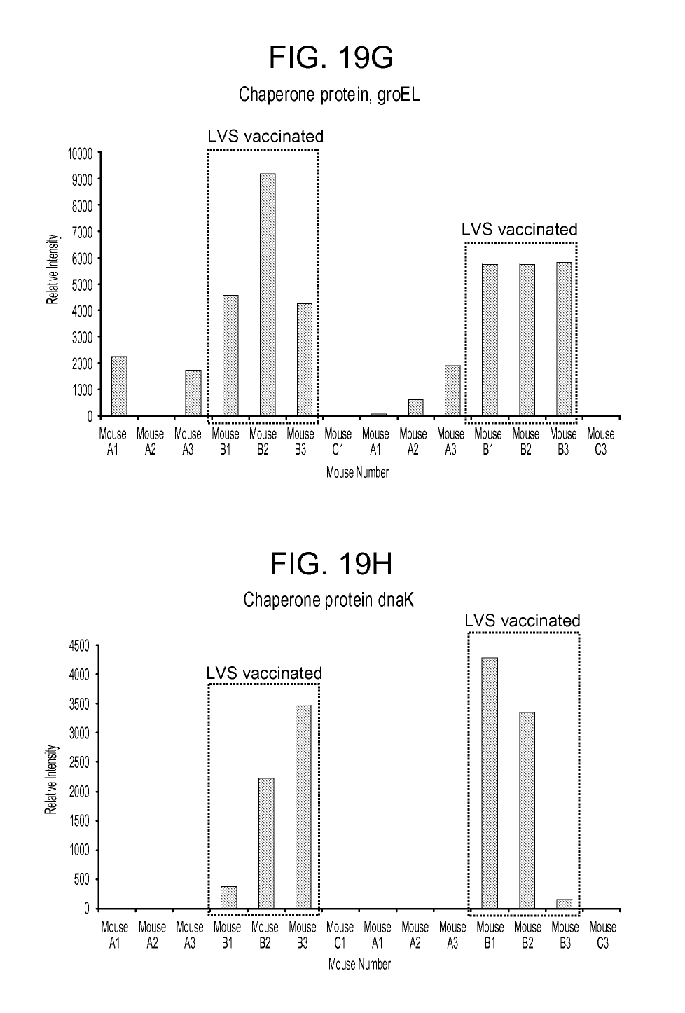

The immunoreactive proteins observed when Western blots were aligned with protein stained 2D-PAGE were very similar to those observed in the other murine immunoproteomics studies described herein. FIG. 18 shows representative blots probed with sera form unvaccinated control mice (FIG. 18A), mice vaccinated SC with LVS (FIG. 18B) and mice vaccinated SC with LVS ΔIgIC (FIG. 18C). Notable differences between the two sets of blots were observed, especially when comparing the observed intensity of individual immunoreactive areas. For example in most murine sera screened, areas of immunoreactivity corresponding to dihydrolipoamide succinyltransferase and 30S ribosomal protein S1 showed much greater intensity of immunoreactivity in LVS-vaccinated mice, compared with LVS ΔIgIC-vaccinated mice. In addition, areas of immunoreactivity corresponding to peroxidase/catalase and GroEL were also observed to have greater intensities of reactivity with sera from LVS-vaccinated mice. Of particular note, immunoreactivity towards the protein DnaK was only observed at detectable levels in sera from LVS-vaccinated mice.

The embodiments and examples described herein are illustrative and are not meant to limit the scope of the invention as claimed. Variations of the foregoing embodiments, including alternatives, modifications and equivalents, are intended by the inventors to be encompassed by the claims. Furthermore, the discussed combination of features might not be necessary for the inventive solution.

### REFERENCES

All patents, patent applications and publications referred to herein and throughout the application are hereby incorporated by reference.
Burke D S. Immunization against tularemia: analysis of the effectiveness of live Francisella tularensis vaccine in prevention of laboratory-acquired tularemia. J Infect Dis 1977 January; 135(1):55-60.
Carlsson H E, Lindberg A A, Lindberg G, Hederstedt B, Karlsson K A, Agell B O. Enzyme-linked immunosorbent assay for immunological diagnosis of human tularemia. J Clin Microbiol 1979 November; 10(5):615-21.
Conlan J W. Vaccines against Francisella tularensis—past, present and future. Expert Rev Vaccines 2004 June; 3(3):307-14.
Dennis D T, Inglesby T V, Henderson D A, et al. Tularemia as a biological weapon: medical and public health management. JAMA 2001 Jun. 6; 285(21):2763-73.
Dienst F T, Jr. Tularemia: a perusal of three hundred thirty-nine cases. J La State Med Soc 1963 April; 115:114-27.
Eigelsbach H T, Hornick R B, Tulis J J. Recent studies o′n live tularemia vaccine. Med Ann Dist Columbia 1967 May; 36(5):282-6.
El Sahly H M, Atmar R L, Patel S M, et al. Safety, reactogenicity and immunogenicity of Francisella tularensis live vaccine strain in humans. Vaccine 2009 Aug. 6; 27(36):4905-11.
Elkins K L, Cowley S C, Bosio C M. Innate and adaptive immune responses to an intracellular bacterium, Francisella tularensis live vaccine strain. Microbes Infect 2003 February; 5(2):135-42.
Ericsson M., Sandström G., Sjostedt A., and Tarnvik A. (1994) Persistence of Cell-Mediated Immunity and Decline of Humoral Immunity to the Intracellular Bacterium Francisella tularensis 25 Years after Natural Infection, J Infect Dis. 170(1): 110-114.
Eyles J E, Unal B, Hartley M G, Newstead S L, Flick-Smith H, Prior J L, Oyston P C, Randall A, Mu Y, Hirst S, Molina D M, Davies D H, Milne T, Griffin K F, Baldi P, Titball R W, Feigner P L. (2007) Immunodominant Francisella tularensis antigens identified using proteome microarray. Proteomics June; 7(13):2172-83.
Feldman K A, Stiles-Enos D, Julian K, et al. Tularemia on Martha's Vineyard: seroprevalence and occupational risk. Emerg Infect Dis 2003 March; 9(3):350-4.
Feigner P L, Kayala M A, Vigil A, et al. A Burkholderia pseudomallei protein microarray reveals serodiagnostic and cross-reactive antigens. Proc Natl Acad Sci USA 2009 Aug. 11; 106(32):13499-504.
Gardy J. L., M. R. Laird, F. Chen, S. Rey, C. J. Walsh, M. Ester, and F. S. L. Brinkman (2005) PSORTb v.2.0: expanded prediction of bacterial protein subcellular localization and insights gained from comparative proteome analysis, Bioinformatics 21(5):617-623
Havlasova J, Hernychova L, Brychta M, et al. Proteomic analysis of anti-Francisella tularensis LVS antibody response in murine model of tularemia. Proteomics 2005 May; 5(8):2090-103.
Havlasova J, Hernychova L, Halada P, et al. Mapping of immunoreactive antigens of Francisella tularensis live vaccine strain. Proteomics 2002 July; 2(7):857-67.
Hornick R B, Eigelsbach H T. Aerogenic immunization of man with live Tularemia vaccine. Bacteriol Rev 1966 September; 30(3):532-8.
Huntley J F, Conley P G, Hagman K E, Norgard M V. Characterization of Francisella tularensis outer membrane proteins. J Bacteriol 2007 January; 189(2):561-74.
Janovska S, Pavkova I, Hubalek M, Lenco J, Macela A, Stulik J. Identification of immunoreactive antigens in membrane proteins enriched fraction from Francisella tularensis LVS. Immunol Lett 2007a Jan. 10.
Janovska S, Pavkova I, Reichelova M, Hubaleka M, Stulik J, Macela A. Proteomic analysis of antibody response in a case of laboratory-acquired infection with Francisella tularensis subsp. tularensis. Folia Microbiol (Praha) 2007b; 52(2):194-8.
Kirimanjeswara G S, Olmos S, Bakshi C S, Metzger D W. Humoral and cell-mediated immunity to the intracellular pathogen Francisella tularensis. Immunol Rev 2008 October; 225:244-55.
Mansfield M A. Rapid immunodetection on polyvinylidene fluoride membrane blots without blocking. Anal Biochem 1995 Jul. 20; 229(1):140-3.
Matyas B T, Nieder H S, Telford S R, Ill. Pneumonic tularemia on Martha's Vineyard: clinical, epidemiologic, and ecological characteristics. Ann N Y Acad Sci 2007 June; 1105:351-77.
Oyston P C, Quarry J E. Tularemia vaccine: past, present and future. Antonie Van Leeuwenhoek 2005 May; 87(4):277-81.
Saslaw S, Carhart S. Studies with tularemia vaccines in volunteers. III. Serologic aspects following intracutaneous or respiratory challenge in both vaccinated and nonvaccinated volunteers. Am J Med Sci 1961 June; 241:689-99.
Saslaw S, Eigelsbach H T, PRIOR JA, WILSON HE, CARHART S. Tularemia vaccine study. II. Respiratory challenge. Arch Intern Med 1961b May; 107:702-14.
Saslaw S, Eigelsbach H T, WILSON HE, PRIOR JA, CARHART S. Tularemia vaccine study. I. Intracutaneous challenge. Arch Intern Med 1961a May; 107:689-701.
Sjostedt A. Family XVII. FRANCISELLACEAE Genus I. Francisella. In: Brenner D J, ed. Bergery's Manual of Systemic Bacteriology. New York: Springer, 2001.
Sjostedt A. Tularemia: history, epidemiology, pathogen physiology, and clinical manifestations. Ann N Y Acad Sci 2007 June; 1105:1-29.
Sundaresh S, Randall A, Unal B, et al. From protein microarrays to diagnostic antigen discovery: a study of the pathogen Francisella tularensis. Bioinformatics 2007 Jul. 1; 23(13):i508-i518.
Tarnvik A. Nature of protective immunity to Francisella tularensis. Rev Infect Dis 1989 May; 11(3):440-51.
Tatusov R. L., et al (2003) The COG database: an updated version includes eukaryotes, BMC Bioinformatics September 11; 4(1):41
Tatusov R. L., Koonin E. V., and Lipman D. J. (1997) A Genomic Perspective on Protein Families, Science October 24; 278:631-7,
Titball R W, Oyston P C. A vaccine for tularaemia. Expert Opin Biol Ther 2003 July; 3(4):645-53.
Titball R W, Petrosino J F. Francisella tularensis genomics and proteomics. Ann N Y Acad Sci 2007 June; 1105:98-121.
Twine S M, Mykytczuk N C, Petit M, Tremblay T L, Conlan J W, Kelly J F. Francisella tularensis proteome: low levels of ASB-14 facilitate the visualization of membrane proteins in total protein extracts. J Proteome Res 2005 September; 4(5):1848-54.
Twine S M, Petit M D, Fulton K M, House R V, Conlan J W. Immunoproteomics analysis of the murine antibody response to vaccination with an improved Francisella tularensis live vaccine strain (LVS). PLoS ONE 2010; 5(4):e10000.
Twine S M, Petit M D, Shen H, Mykytczuk N C, Kelly J F, Conlan J W. Immunoproteomic analysis of the murine antibody response to successful and failed immunization with live anti-Francisella vaccines. Biochem Biophys Res Commun 2006 Aug. 4; 346(3):999-1008.
Viljanen M K, Nurmi T, Salminen A. Enzyme-linked immunosorbent assay (ELISA) with bacterial sonicate antigen for IgM, IgA, and IgG antibodies to Francisella tularensis: comparison with bacterial agglutination test and ELISA with lipopolysaccharide antigen. J Infect Dis 1983 October; 148(4):715-20.

1-3. (canceled)

4. A biomarker for tularemia, consisting essentially of Acetyl-CoA.

5. The biomarker of claim 4, which is reactive with both patient and vaccinee sera at a minimum frequency of 30%.

6. A method of evaluating immunity in a subject against Francisella tularensis, comprising:
a. contacting serum from the subject with the biomarker of claim 1;
b. evaluating immunoreactivity of the serum to the biomarker;
c. determining the protection against tularemia based on immunoreactivity to the biomarker.

7. A method of evaluating efficacy of a vaccine for Francisella tularensis, comprising:
a. contacting serum from a vaccinated model of tularemia with the biomarker of claim 1;
b. evaluating immunoreactivity of the serum to the biomarker;
c. correlating the immunoreactivity to immunoprotective status of the model; and
d. predicting vaccine efficacy in human based on the correlation of step c.

8. A method for evaluating the potential effectiveness of a vaccine for Francisella tularensis in inducing an immune response against Francisella tularensis, comprising detecting the presence or absence in the vaccine of Francisella tularensis protein FTT_{—0472} (Acetyl-CoA carboxylase biotin carboxyl carrier protein), the presence of which is predictive of induction of an immune response.

9. The method of claim 7, wherein the vaccine is Live Vaccine Strain vaccine.

10. The method of claim 8, wherein the vaccine is Live Vaccine Strain vaccine.
